(19) United States
(12) Patent Application Publication
(10) Pub. No.: US 2022/0192780 A1
(43) Pub. Date: Jun. 23, 2022

(54) SYSTEMS AND METHODS FOR INTRAOPERATIVE ISOLATION AND CONTROL OF SURGICAL SITE ENVIRONMENTS

(71) Applicants: Stephen Michael OKAJIMA, Boston, MA (US); Debbie Lin TEODERESCU, Cambridge, MA (US); Mike Horia Mihail TEODERESCU, Cambridge, MA (US); SURGIBOX INC., Cambridge, MA (US)

(72) Inventors: Stephen Michael Okajima, Boston, MA (US); Debbie Lin Teodorescu, Cambridge, MA (US); Mike Horia Mihail Teodorescu, Cambridge, MA (US)

(73) Assignee: SURGIBOX INC., Cambridge, MA (US)

(21) Appl. No.: 17/601,989

(22) PCT Filed: May 10, 2020
(86) PCT No.: PCT/US2020/032280

§ 371 Date: Oct. 7, 2021

Related U.S. Application Data
(60) Provisional application No. 62/845,843, filed on May 9, 2019.

(51) Int. Cl. A61B 90/40; A61G 10/00; A61B 46/27

(52) U.S. Cl. CPC A61B 90/40; A61B 2090/401; A61B 46/27; A61G 10/005

(57) ABSTRACT

A portable surgical system including a transparent and flexible plastic enclosure is disclosed. The enclosure is attached reversibly to the patient's body encompassing the surgical site such as to isolate and regulate the immediate environment of the surgical site, and to reduce bodily fluid splatters from the surgical site to the surgical providers. The enclosure includes an environmental control system configured to actively monitor and control the environment inside the enclosure such as to create and maintain essentially sterile conditions. The surgical systems can be used both for surgeries on a torso of a patient and on surgeries on arms or legs. The portable surgical system is lightweight and portable and can be used in con-ventional operating rooms to improve sterility, or in other circum-stances where no operating room is available, such as field hospitals and for on the field surgeries.

### CROSS REFERENCE TO RELATED APPLICATIONS

The present application is a continuation-in-part of U.S. application Ser. No. 16/317,892 filed on Jan. 15, 2019. The present application claims priority from the U.S. Provisional Patent Application No. 62/845,843 filed on May 9, 2019 and titled “System and methods of exhausting airflow and contaminants from within an environment”.

The following applications are incorporated hereinafter in their entirety as if full set forth herein: PCT application PCT/US17/42266 filed on Jul. 14, 2017 and titled “Ultraportable System for Intraoperative Isolation and Regulation of Surgical Site Environments”; U.S. Provisional Patent Application No. 62/362,893 filed on Jul. 15, 2016 and titled “Modular Surgical Suite”; PCT application PCT/US19/32148 filed on May 14, 2019 and titled “Sterile Sleeves for Portable Surgical Systems”; and International PCT application PCT/US2019/051502 filed on Sep. 17, 2019 and titled “Data Analytics and Interface Platform for Portable Surgical Enclosure”.

### BACKGROUND OF THE INVENTION

### I. Field of the Invention

Exemplary embodiments of the present invention relate to a portable surgical system for regulating intra-operative environments over surgical sites; and to methods for implementing and using the same.

### II. Discussion of the Background

Over 25% of the global disease burden requires surgical therapy, which could prevent over 18 million deaths per year. These range from obstetric complications to traumas to infections to cancer and beyond. Yet 2 billion people have no meaningful access to safe surgical care, and 2-3 billion more have access only to unsterile surgeries in contaminated environments, leading to disproportionate rates of surgical infections. Innovations in this field typically focus upon making operating rooms and operating room ventilation systems more mobile, such as in tent format. However, such systems remain costly to purchase and to maintain. Moreover, such systems are difficult to transport rapidly to remote areas. At the same time, over 85,000 medical providers are infected by patient bodily fluids annually, with 90% of infected providers worldwide having been exposed while working in low-resource settings. While personal protective equipment mitigates these risks to some extent, there is a definite trade-off between the level of protection and both the cost as well as the user comfort, which is well-documented to correspond to user compliance. Thus, there is a need for systems and methods minimizing the intraoperative exposure to infectious risks, keeping of a clean operative environment, while maintaining the functionalities needed for performing safe surgical procedures.

Exemplary embodiments of the present invention aim to address both challenges of patient and provider intraoperative exposure to infectious risks by implementing an ultraportable system for intraoperative isolation and regulation of surgical site environments. The systems and methods enable the providing of self-contained, passive and active, bilateral barrier against exchange of contaminants between incisions and the greater surgical area. The systems may be enabled to provide a clean operating environment, proper airflow, proper pressurization, and proper disposal of contaminants. The system provides the access and functionalities necessary to performing safe surgical procedures.

The above information disclosed in this Background section is only for enhancement of understanding of the background of the invention and therefore it may contain information that does not form any part of the prior art.

### SUMMARY OF THE INVENTION

Exemplary embodiments of the present invention provide a portable surgical system for regulating intra-operative environments over surgical sites.

In an exemplary embodiment it is disclosed a portable surgical system including a flexible enclosure separating a surgical environment inside the enclosure from an user environment outside the enclosure. The enclosure may include one or more areas of high optical clarity for viewing the inside of the enclosure. While the surgical system is deployed in use, only the surgical site is included within the surgical enclosure, and the remainder of the patient body is essentially excluded from the surgical environment inside the enclosure. The portable surgical system may further include an environmental control system configured to supply air to the enclosure such as to create essentially sterile conditions inside the enclosure and one or more ports for accessing the surgical site. The enclosure may further include an exhaust system configured to eliminate air from the enclosure. The exhaust system includes one or more exhaust-channels disposed on the side and along the enclosure. The exhaust-channels may further include a plurality of holes disposed on one or more lines along the exhaust-channels, the holes being configured to allow air to flow from the enclosure into the exhaust-channels and further into the environment outside the enclosure.

In an exemplary embodiment the exhaust-channels may include one or more exhaust-valves configured to adjust the airflow magnitude into the exhaust-channels. The exhaust-channels may include one or more exhaust-valves configured to adjust the airflow magnitude into the exhaust-channels. In an exemplary embodiment the environmental control system is configured to adjust the airflow magnitude into the air-supply-tube via a supply-valve. In an exemplary embodiment, the air-supply-tube may include one or more flow-guides, wherein each of the flow-guides is disposed on top of a corresponding perforation such as to guide the flow of air from the air-supply-tube into the enclosure. Each of the flow-guides may include a mini-valve configured to control the magnitude of the airflow through the corresponding perforation on which the flow-guides is disposed on. The environmental control system may to control the mini-valves via electrically controlled actuators.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 is a side view of an inflated portable surgical enclosure adhered to the patient's torso surgical site via incise drape, with air inflow from air supply in enclosure side closest to patient feet, directed in cranial longitudinal direction over the patient's surgical site.

FIG. 2 is a top view of the inflated portable surgical enclosure from FIG. 1 with two users working via arm ports in operating-section on the torso surgical site, and two users working via arm ports in instrument-section.

FIG. 3 is a side view of an alternate embodiment of the surgical enclosure which utilizes a central frame and oblique tethers in cranial and caudal directions to assist with holding up the enclosure.

FIG. 4 is an axial view perpendicular to the view illustrated in FIG. 3 showing the shape of the central frame and the tethers to support it. Patient, instrument tray, and ports are excluded from illustration.

FIG. 5 is a side view of an additional alternative embodiment which utilizes two vertical frames at each of the cranial and caudal ends of the enclosure, and tethers to support the surgical enclosure.

FIG. 6 is an axial view perpendicular to the view illustrated in FIG. 5 showing the shape of one of the two identical frames and the tethers which support the enclosure.

FIG. 7 is a side view of the embodiment shown in FIG. 5 and FIG. 6 demonstrating how the frame and tethers prevent the enclosure from collapsing on the surgical site in the case of sudden pressure loss.

FIG. 8 is an axial view perpendicular to the view illustrated in FIG. 7.

FIG. 9 is a side view of an alternate embodiment of the surgical enclosure and frame, in 110 which the rigid frame fully supports the enclosure with frame attachment to each of the sides defining the top of the enclosure. The enclosure extends circumferentially around the patient torso.

FIG. 10 shows an oblique perspective view of the frame and plastic enclosure shown in FIG. 9.

FIG. 11 is a schematic of the portions of the air supply system external to the enclosure.

FIG. 12 is an alternate embodiment for the air supply system which incorporates a back-up manual pump.

FIG. 13 shows the axial view with the overhead inlet tube valve in the enclosure open during active air inflow, signaling adequate flow.

FIG. 14 shows the axial view with the tube valve FIG. 13 pinched closed by the enclosure's positive pressure, thus sealing the system and preventing backflow.

FIG. 15 shows an exemplary embodiment of the material ports.

FIG. 16 shows an alternate embodiment of the material ports with different port sizes.

FIG. 17 shows an alternate embodiment of the material ports, in which there is a small port above each set of sleeves and a larger port in the middle.

FIG. 18 shows an alternate embodiment of the material port, in which a bimodal port can be opened either fully or only partially depending on the need.

FIG. 19 is a side view at the level of the arm port, showing user sleeves and gloves in an inflated enclosure are pinched together by the positive pressure in the surgical enclosure prior to their use.

FIG. 20 is a schematic view of the airflow within the enclosure as traveling through the valve system continuously into and through the manifold system, with perforations varying in density along the manifold to produce uniform flow.

FIG. 21 is a schematic of a manufacturing process to produce the embodiment of FIG. 20.

FIG. 22 is a graph relating manifold perforation density and air exit velocity from the embodiment of FIG. 20.

FIG. 23 is a schematic view of the airflow within the enclosure as traveling through the valve system continuously into and through the manifold system, with perforations varying in diameter along the manifold to produce uniform flow.

FIG. 24 is a schematic sample setup workflow for the frame embodiment described in FIGS. 3 and 4.

FIG. 25 is a schematic sample setup workflow for the frame embodiment described in FIG. 9.

FIG. 26 shows a graph of the particle concentration inside the enclosure as function of environment parameters as obtained from tests on a prototype portable surgical system.

FIG. 27 shows an exemplary embodiment of a sleeve.

FIG. 28 shows two stages of a method of accessing the enclosure via a sleeve.

FIG. 29 shows an isometric view of a portable surgical system including an air supply tube, exhaust channels and fluids elimination channels.

FIG. 30 shows a side view of a portable surgical system including an air supply tube, exhaust channels and fluids elimination channels.

FIG. 31 shows a top-down view of a portable surgical system including an air supply tube, exhaust channels and fluids elimination channels.

FIG. 32. shows a cross-sectional view of a portable surgical system including an air supply tube, exhaust channels and the airflow inside the enclosure.

FIG. 33 shows a cross-sectional view of a portable surgical system including a fluids elimination channels and arrows indicating the collection of fluids into the elimination channels.

FIG. 34 shows a cross-sectional view of a surgical enclosure showing the directionality of the airflow from the inlet tube towards the exit holes of the exhaust channels.

FIG. 35 shows a directional airflow cylinder and flow guide and a divider within the inflation tube.

FIG. 36 shows the directional airflow cylinder and flow guide including a mini-valve.

FIG. 37 shows exemplary embodiments of directional airflow elements included in the airflow inflation tube.

FIG. 38 shows an embodiment of a directional airflow cylinder and flow guide including a mechanical actuator and flap.

FIG. 39 shows an exploded view of the surgical enclosure including the flow guide cylindrical directional airflow element.

FIG. 40 shows an embodiment of the surgical enclosure with actuated valves and an control unit communicating with sensors in the enclosure and with the actuated valve elements.

FIG. 41 shows a view of a flow guide including actuated valves symmetrically positioned in a shut position on either side of the divider wall.

FIG. 42 shows diagram of an environment monitoring and control system for the environment inside the surgical enclosure.

FIG. 43 represents a cross-sectional view of the surgical enclosure connected to an environment control unit with a pump connected to an inflation tube and a sensor sampling tube.

### DETAILED DESCRIPTION OF THE ILLUSTRATED EMBODIMENTS

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

It will be understood that when an element or layer is referred to as being “on” or “connected to” another element or layer, it can be directly on or directly connected to the other element or layer, or intervening elements or layers may be present. In contrast, when an element or layer is referred to as being “directly on” or “directly connected to” another element or layer, there are no intervening elements or layers present. It will be understood that for the purposes of this disclosure, “at least one of X, Y, and Z” can be construed as X only, Y only, Z only, or any combination of two or more items X, Y, and Z (e.g., XYZ, XY, YY, YZ, ZZ).

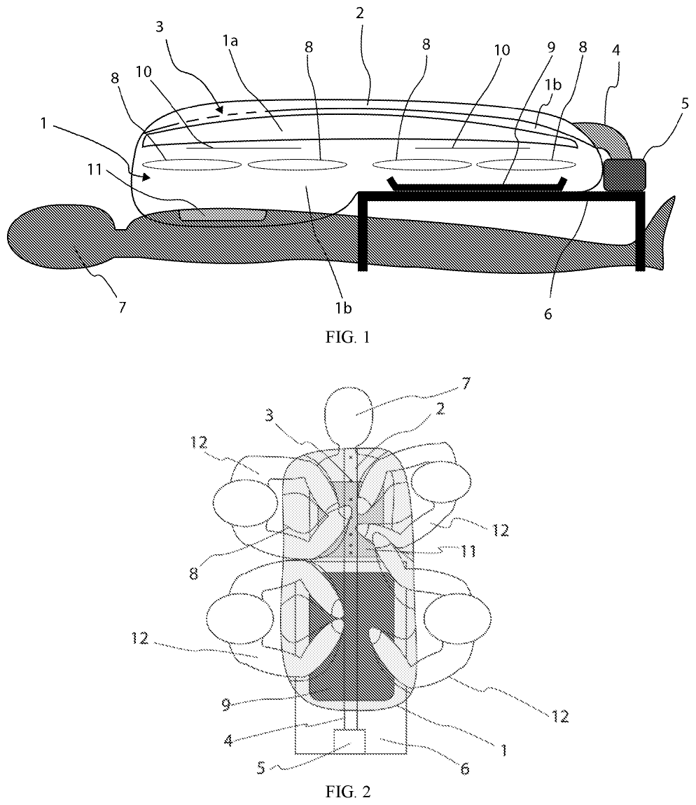

FIG. 1 illustrates a preferred embodiment of a portable surgical system. The portable surgical system includes a flexible plastic enclosure 1 configured to be supplied with air under positive pressure via an environmental control system 5. The enclosure 1 may be adhered to a surgical site of a patient 7 via an incise drape 11 as shown in FIG. 1. The incise drape may be a flexible plastic drape and may include a removable skin adhesive on one side, with or without antimicrobial impregnation. The portable surgical system may be configured such that filtered air is blown or passed through a longitudinal tubular valve with walls of flexible, collapsible plastic such as polyethylene 2 and through a manifold with perforations 3. The filtered air may be blown such as to cause an essentially uniform laminar air flow onto the surgical site and through the enclosure.

The portable surgical system may include a plurality of ports, such as arm ports 8 and material ports 10 shown in FIGS. 1 and 2. In an exemplary embodiment the portable surgical system may include four pairs of integrated, cuffed sleeves in the arm ports 8. The ports 8 provide users with access to the inside of the enclosure, as shown in FIG. 2. The material ports 10 may be used to move the surgical tray 9 to the inside of the enclosure 1 prior to the surgical procedure. The portable surgical system may further include an instrument tray holder 6 which may be placed around the legs of the patient 7. The tray 9 may be disposed on top of the instrument tray holder 6.

In the preferred embodiment shown in FIG. 1, the perforations which define the manifold outlets 3 in the overhead tube decrease in density along the remainder of the manifold over the operating-section such that the airflow over the incise drape 11 is essentially constant. If the environmental control system 5 is shut off, the flexible overhead tube 2 is pinched shut, thus 215 sealing the enclosure 1 and preventing backflow into the fan and filter 5.

The portable surgical system may include a surgical enclosure, a frame, and an environmental control system.

A. Structure of Surgical Enclosure

In an exemplary embodiment the surgical enclosure may be disposable, such as the enclosure 1 shown in FIG. 1. In an exemplary embodiment the surgical enclosure may be supplied folded like a surgical gown. When set up, the surgical enclosure may comprise one or more top view panels of optically-clear plastic 1a, such as polyvinyl chloride. The remainder of the surgical enclosure sides may comprise a flexible, impermeable plastic, such as low-density polyethylene. The sides of the instrument-section may be shorter than those of the operating-section, in order to fit over an instrument tray holder. In the preferred embodiment shown in FIG. 1, the bottom of the enclosure is continuous with the sides.

The panel of incise drape 11 may be incorporated into the bottom of the operating-section as shown in FIG. 1. The incise drape serves as the interface with the patient body. The size and shape of the incise drape 11 may be configured to cover the surgical site on the patient's body while essentially excluding body surface outside the surgical site. Consequently, as seen in FIG. 1, only the surgical site of the patient's body (i.e. area covered by the incise drape 11) is included within the surgical enclosure, while the remainder of the patient body is excluded from the sterile field. By excluding from the surgical enclosure the unnecessary body surface, the efficacy of the system is significantly improved since the patient's body surface contributes to environment contamination inside the enclosure. In particular, the exclusion of high-contaminant regions such as the oropharynx or the genitals is likely to significantly improve the efficacy of the system. The surgical enclosure 1 may include incise drapes 11 of different shapes and sizes and may be disposed at different positions on the surgical enclosure such as to fit the needs of different types of medical procedures. The bottom corners of the surgical enclosure may include straps for securing the enclosure to the patient or to the operating table for additional stability.

FIGS. 9 and 10 illustrate a side view and a perspective view, respectively, of a second preferred embodiment of the portable surgical system. In the second preferred embodiment the portable surgical system includes an incise drape-less surgical enclosure 1 wherein the operating-section of the patient is placed inside the enclosure and wherein the bottom of the enclosure remains continuous with the sides at the level of the instrument-section. In the operating-section of the enclosure, one side of the enclosure may be elongated so as to enable tucking under the patient body, thereby eliminating the continuous bottom panel. After passing under the patient body, the residual length of the elongated side may be secured to the contralateral enclosure side along the free edge of the elongated side. The cranial end of the operating-section 18 as well as the interface with the instrument-section 18 may be secured against the patient via integrated straps.

Embodiments of the invention are described herein with reference to figures and illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

The portable surgical systems disclosed herein may include alternate or additional sections which could be added based on procedural needs, such as to accommodate additional instrument trays or users. The above embodiments presented in this disclosure merely serve as exemplary embodiments and it will be apparent to those skilled in the art that various modifications and variations can be made in the present invention without departing from the spirit or scope of the invention.

B. Structure of Frame

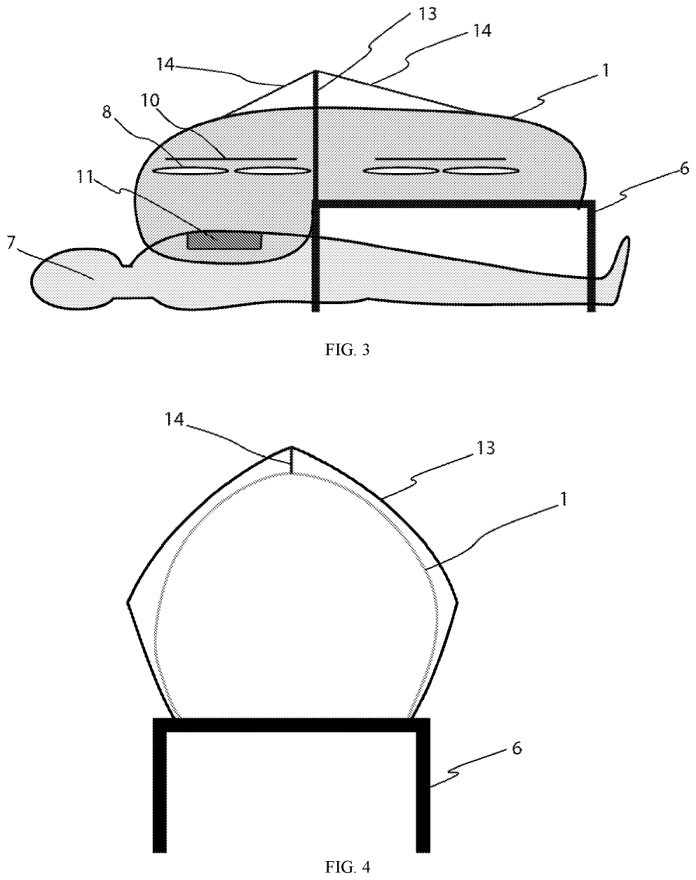

In an exemplary embodiment, illustrated in FIGS. 3 and 4, the portable surgical system may include a central frame 13 and tethers 14 intended to support the enclosure 1 in the case of a sudden pressure loss. The central frame 13 may be lightweight and/or collapsible so as to be easily transported. The frame may be made of a rigid material, such as plastic, rigid polyvinyl tubes, aluminum tubing, and other materials familiar to practitioners knowledgeable in the field. The frame may include four oblique tubes which are reversibly secured to the instrument tray holder or operating table such that the instrument tray holder or operating table form the bottom of a pentagon when viewed axially as in FIG. 4. One or more of these pieces may be connected to one another via custom connectors or hinges, configured to maintain the pentagon within the same plane. The topmost vertex of the frame may be reversibly attached to the disposable component top, such as via a formed plastic slot in the disposable component or via tether 14 only. Tethers 14 may support the plastic enclosure 1 directly underneath the frame 13, as shown in FIG. 4, as well as longitudinally over the incise drape 11 and instrument tray holder 6. Frame 13 and tethers 14 are configured to provide support to the enclosure 1 in the event of a sudden pressure loss. Various other tether arrangements may be utilized to optimize support from the central frame, depending on system requirements.

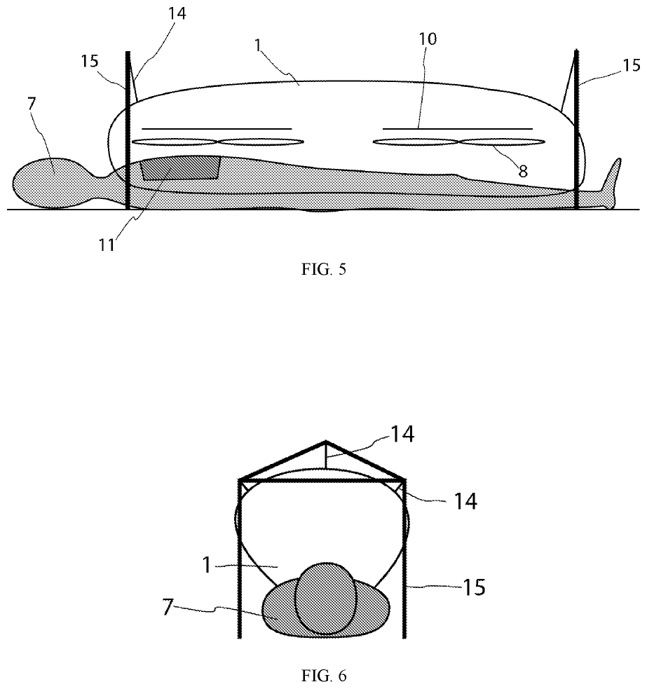

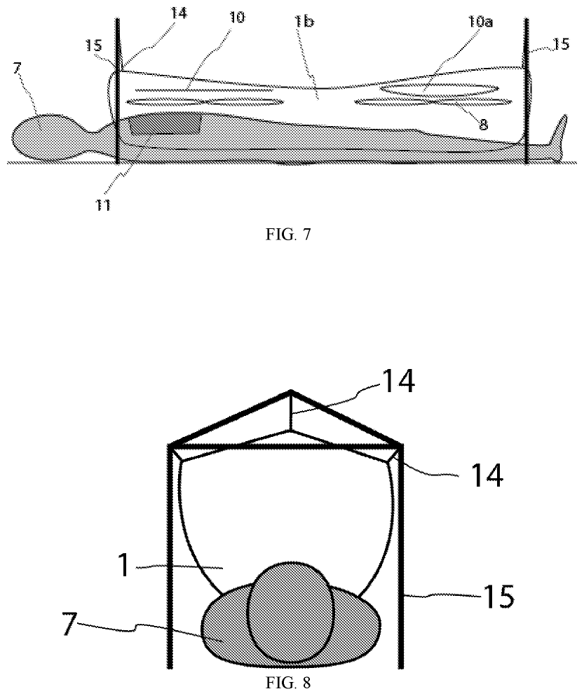

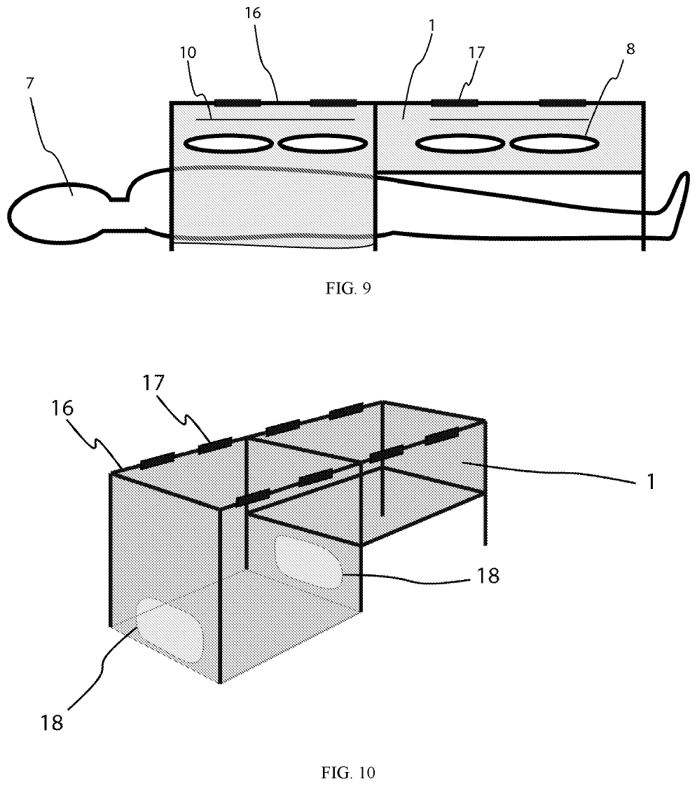

In another exemplary embodiment the portable surgical system may include a frame 15 and tethers 14 as illustrated in FIGS. 5-8. Frame 15 and tethers 14 are configured such as to provide support to the enclosure 1 in the event of a sudden pressure loss. Instead of supporting the surgical enclosure centrally, frame 15 includes two vertical sections disposed at the cranial and caudal ends of the enclosure. FIG. 5 provides a side view of the frame 15 and tethers 14, and FIG. 6 provides a front view of the same system. FIGS. 7 and 8 show how the frame 15 and tethers 14 support the deflated enclosure 1b in the case of a sudden pressure loss, resulting from, for instance, an open port 10a.

In an exemplary embodiment the portable surgical system may include a collapsible, rigid frame 16 and a flexible plastic enclosure 1 as illustrated in FIGS. 9 and 10 and as described in the section “Structure of Surgical Enclosure” paragraph 3 in which the surgical enclosure 1 encloses the patient's 7 torso. The portable surgical system according to this embodiment does not require a separate instrument tray holder. The enclosure 1 is reversibly sealed at the patient's suprapubic region and axillae via adjustable opening 18. This embodiment does not structurally rely on positive pressure to the extent that the previous embodiments, illustrated in FIGS. 1-8, do. The frame may comprise six vertical pieces forming the edges of two connected partial cuboids, reversibly attached to under the patient or to the operating table. As seen in FIGS. 9 and 10, the frame may include two pieces at the cranial end, two at the caudal end, and two at the junction between the operating and instrument sections. These pieces may incorporate telescoping function to accommodate different patient body sagittal abdominal diameters. These vertical pieces may be connected as shown in FIG. 9, with three pieces horizontally at the top and two additional horizontal pieces defining the instrument tray section; these latter two pieces are at a level above the patient where desired for an instrument tray holder. The frame may further include two longitudinal pieces, perpendicular to both of the above types, forming the operating section; and two additional longitudinal pieces forming the instrument section. One or more of all of these pieces may be connected via hinges or custom connectors. The enclosure may be connected to the frame reversibly 17 in such manner as to place uniform outward tension on the top view panel.

In an alternate embodiment, the frame of FIGS. 9 and 10 may be comprised of deformable materials that can be activated to a rigid shape through application of heat or a current in order to allow for more compact packaging during transportation.

C. Ports

The various embodiments of the portable surgical system may have surgical enclosures which include a plurality of ports. The enclosure may include two major types of ports. The first type of port on the enclosure is arm ports 8, as shown in FIGS. 1, 2, 3, 5, 7, and 9, which allow access to the inside of the enclosure by either provider arms or augmenting instrumentation taking the place of arms such as laparoscopes or robots.

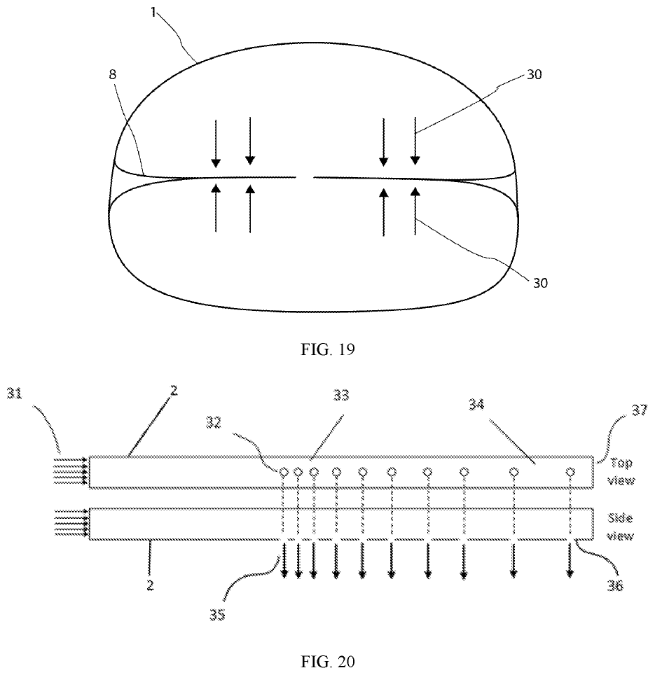

The number of arm ports is dependent on procedural need. The preferred embodiments illustrated in FIGS. 1, 2, 3, 5, 7, and 9 include four pairs of arm ports 8, two on each side of the enclosure 1. Depending on use scenario, the arm ports may take three major forms. The first form for the arm port is a simple opening in the side of the enclosure which seals reversibly against user arms. The second form for the arm port is a sleeve as shown by 8 in FIG. 2, which is a hollow cylinder or frustrated cone of impermeable plastic that tapers toward the inside of the enclosure away from the wall. The length of the sleeve is adequate to permit ergonomic handoff of instruments among ports at contralateral ends of the system. The material of the sleeve may be the same as the one used for the enclosure side, or it can be a different one, such as a material used in surgical gown sleeves. The sleeve end may be free or may incorporate a cuff of elastic material to fit against the user wrist. The third form for the arm port is the same as the second form, but ending in a glove. FIG. 19 shows a side view at the level of the arm port, showing user sleeves and gloves in an inflated enclosure. The user sleeves and gloves are pinched together by the positive pressure in the surgical enclosure prior to their use.

The second type of port on the enclosure is a materials port 10, as shown in FIGS. 1, 3, 5, 7 and 9, which allows the instrument tray 9 and instruments to be moved into the enclosure 1 prior to the procedure. Additionally, the port allows materials to be moved in and out of the enclosure throughout the surgical procedure. In the case of a caesarean section, it is imperative that the newborn child can be quickly and ergonomically passed out of the enclosure so it can receive care.

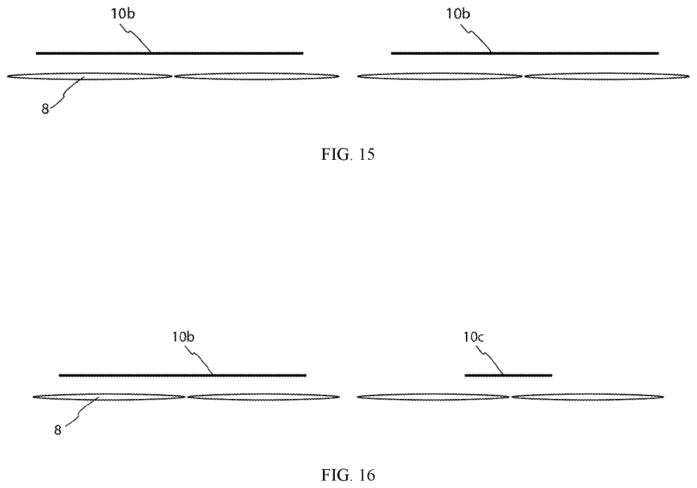

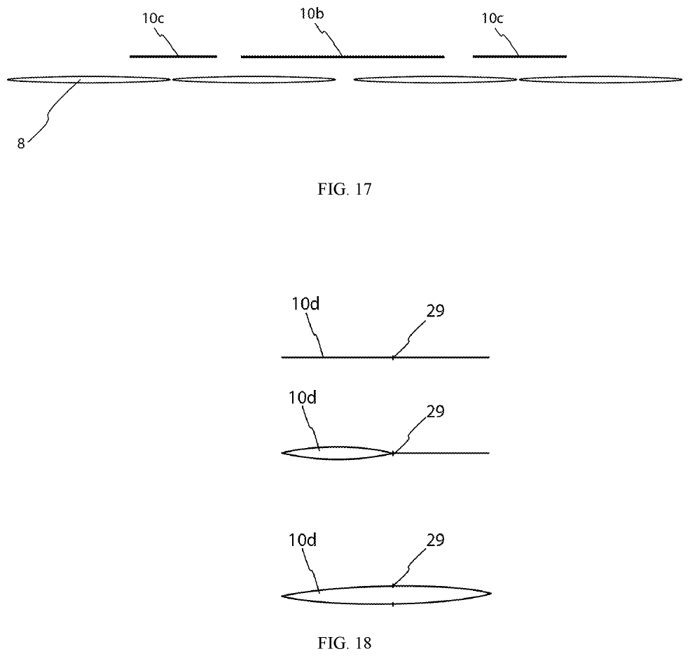

FIGS. 15-17 show various possible configurations of the ports, although additional embodiments would be conceived of that fit the nature of the claims. In an exemplary embodiment, the enclosure 1 may include large ports 10b as shown in FIG. 15, small ports 10c as shown in FIGS. 16 and 17, or both large ports 10b and small ports 10c as shown in FIGS. 16 and 17. Small ports 10c are configured such that small items may be passed in or out of the enclosure without significant relative loss of enclosure volume or pressure, regardless of frame availability, because the Environmental Control System (e.g. a fan) can increase the gas inflow to match the outflow. Large ports 10b permit the moving of large items like the instrument tray, neonates, et cetera in and out of the enclosure. FIG. 18 shows an exemplary embodiment of the port, in which a connector 29 splits a port in half, allowing it to act as a small port or large port. This bimodal port 10d ensures that any user can have access to both a small port and a large port. In addition to episodic access for large items, the ports can also provide ongoing access for lines, tubes, wires, and drains requiring access to external resources. The connector 29 may be a zipper slider that slides over the zipper teeth rows thereby adjusting the size of the port. Alternatively, it can be a material such as hook and loop fastener or magnets which provide rapidly reversible attachment. There are a number of ways the materials ports can be implemented. They must be easy to open and close repeatedly, such as can be achieved through the use of magnetic strips, hook-and-loop fasteners, plastic zippers, flexible inflatable tubes compressed against one another, or other methods.

D. Environmental Control System

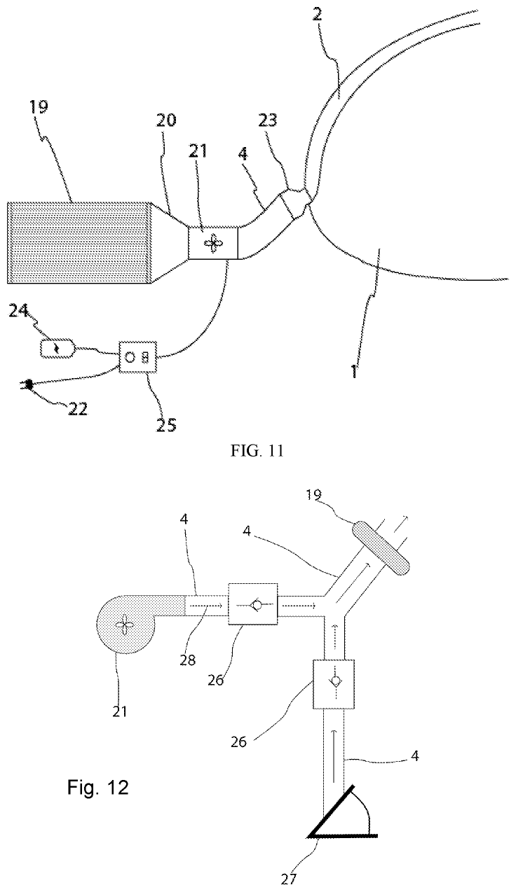

The portable surgical system includes an environmental control system. In a preferred embodiment, as the one shown in FIG. 11, the environmental control system may include a HEPA filter 19, fan (blower with motor) 21, filter-blower adapter 20, battery 24, and control section 25, connected to the enclosure via sterile flexible tubing 23. These external components (i.e. components 19, 20, 21, 23, 24, and 25) are collectively referred to as air supply system. The battery 24 may be disposable or rechargeable, and the system can also run off the electrical grid 22 if the procedure occurs in a setting in which this is possible. The air supply system may be connected to the flexible overhead tube 2 of the surgical enclosure with flexible tubing so that the inlet height of the overhead airflow tube 2 can adjust based on the level of inflation of the enclosure 1. The HEPA filter immediately downstream of air inflow may be changeable and customizable such that it provides one or more other controls based on procedural need, such as humidity modulator filter, gas content with supply of medical gases, or temperature modulator with heat/cold sinks.

In an alternative exemplary embodiment, the air supply system includes both an electrical fan 21 as well as a manual pump 27 as illustrated in FIG. 12. The manual pump 27 provides redundancy and may be used in the event of unavailability of electrical power supply or to provide higher flows without expending electrical power. The manual pump can be implemented in any number of mechanical setups familiar to practitioners in the art, including but not limited to via manual or pedal bellows-style pump or other general positive displacement pump, or manual or pedal rotary pump. The air supply system may further include one or more one-way valves 26 which allow the air from either only the electrical fan 21 or only the manual pump 27 to flow toward the plastic enclosure. The filter 19 is downstream of both electrical and manual air supply.

The external air supply system connects to the enclosure. In an exemplary embodiment, the air is supplied through an inlet and thereby blows through the entire enclosure cranially to caudally. Airflow adequacy may be checked by timing of inflation of the surgical enclosure 1 or by the rising of a windsock in the enclosure embodiment shown in FIG. 9. The windsock may include a short tube of flexible plastic of the same material as the enclosure side. In another exemplary embodiment, the inlet is connected to a horizontal manifold running side to side over the patient. The manifold may include an additional fold of the enclosure side plastic which is sealed together into tubular structure and perforated 3 to create parallel, uniform streams of laminar air outflow into the enclosure.

In a preferred exemplary embodiment the inlet is connected to a flexible tube, such as the overhead flexible tube 2 shown in FIGS. 1 and 2. The flexible tube 2 may include a plurality of perforations 3 acting as manifold. The flexible tube may run side to side or along the enclosure. The flexible tube may be formed by sealing a fold of the enclosure into a tubular structure. The flexible tube may be a collapsible tube that opens when air is blown into the enclosure and closes when air moves out of the enclosure such that transmural pressure from the enclosure favors tube collapse.

In a preferred exemplary embodiment, the flexible tube may include a plurality of perforations 3 disposed such as to create parallel, uniform streams of laminar air outflow into the enclosure. Uniform airflow is accomplished in our preferred embodiment, as described by the design and manufacturing implementations detailed in FIGS. 20-22, by varying the density of perforations in the collapsible tube in which the density of perforations is higher at the end of the tube closer to the supply of the air 31 and the density of perforations decreases as the distance from the supply increases until the density is at its lowest value at 37.

Inventors in this application came to the realization that nearly uniform air flow may be accomplished when the perforation density along the tube decreases according to the inverse of an elliptically shaped function. Starting from the observation that the pressure within an inviscid flow will rise along a streamline if the velocity of the airflow decreases, inventors of this application have found that in a perforated tube of constant cross sectional area, the velocity within a tube will drop as it passes perforations from which flow is emanating, as long as the flow is of nearly constant density which will be the case for flows of air substantially below the speed of sound. Further, inventors have come to the realization that the pressure in a perforated tube rises as the distance from the source increases and, as a result, the rate of flow from each perforation rises with distance from the source assuming the perforations are of constant cross sectional area. As shown in FIG. 20, the velocity is low 35 at locations close to the source 31 and the velocity is high 36 at locations far from the source 31. If the density of perforations were uniform, the flow of air would be too large at locations far from the source and too small at locations nearer to the source.

An exemplary embodiment of the invention discloses a flexible tube 2 (as shown by FIGS. 1, 2, 11, and 20) including a plurality of perforations disposed at such positions (x₁, x₂, x₃, x₄, . . . xₖ) along the tube as to create uniform air flow. The exemplary embodiment in FIG. 20 illustrates a tube including a plurality of perforations disposed in a single axial row along the tube. The tube may include multiple axial rows of perforations disposed on the circumference of the tubes such as to cover the entire surface of the tube or only a certain desired region, such as the region facing towards the surgical site. The multiple axial rows may be essentially parallel with each other and with the axis of the tube.

The perforations are disposed along the flexible tube such that the axial positions of the perforations along the flexible tube may follow a mathematical relation (x₁, x₂, x₃, x₄, . . . xₖ)=Φ(V, d, D, ρ, k, L), where V is the air velocity from the source, D is the diameter of the tube, d is the diameter of the perforations, and ρ is an air density, L is the length of the perforated section, and k the number of perforations in a row. The mathematical relation Φ(V, d, D, ρ, k, L) is determined as explained hereinafter.

The positions of the perforations along the flexible tube may be expressed by a plurality of mathematical expressions: x₁=Φ₁(V, d, D, ρ, k, L); x₂=Φ₂(V, d, D, ρ, k, L); x₃=Φ₃(V, d, D, ρ, k, L); xₖ=Φₖ(V, d, D, ρ, k, L); where V is the air velocity from the source, D is the diameter of the tube, d is the diameter of the perforations, and p is an air density. The mathematical expressions Φ₁(V, d, D, ρ, k, L), Φ₂(V, d, D, ρ, k, L) . . . Φₖ(V, d, D, ρ, k, L) are determined as explained hereinafter and may be closed form expressions of (V, d, D, ρ, k, L).

The specific form of the perforation density needed for uniform air flow can be determined by an iterative computation.

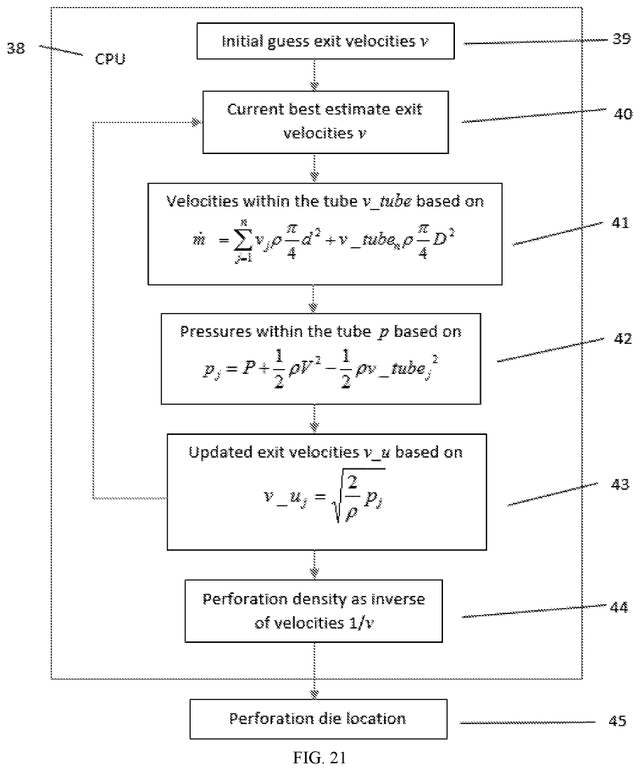

The iterative computation may include a plurality of iterations, wherein each iteration includes a plurality of steps as described in FIG. 21. Within a CPU 38, begin with an assumed form of the exit velocities 39 such as a linearly increasing distribution. These assumed exit velocities will be denoted as vⱼ with a unique subscript for each of the many holes numbered j=1 to k (i.e. velocities v₁, v₂, v₃, . . . vₖ shown in FIG. 20 corresponding to perforations 1, 2, 3 . . . k).

In a first step of the first iteration (see 40 in FIG. 21) it is assumed a form of the exit velocities 39. The assumed exit velocities (i.e. v₁, v₂, v₃, . . . vₖ) may be estimated as a linearly increasing distribution such as

$${v}_{j} = V \cdot ( \frac{{D}^{2}}{k \cdot {d}^{2}} ) \cdot ( j - 1 ) ,$$

where V is the axial air velocity at the source, D is the diameter of the tube, d is the diameter of the perforations, k is the number of perforations, and j is the index of the perforation or hole.

In a second step of the first iteration (see 41 in FIG. 21) the exit velocities (v₁, v₂, v₃, . . . vₖ) estimated at 40 are used to compute an estimate of the velocities within the tube v_tube 41 (i.e. v_tube₁; v_tube₂; v_tube₃; . . . ; v_tubeₖ). The velocity v_tubeₙ is the axial velocity inside the portion of the tube between perforation “n” and perforation “n+1”. Mass conservation requires that for any hole number n in a tube of diameter D with perforations of diameter d the following Equations are satisfied:

$$\dot{m} = {\sum }_{j = 1}^{n} {v}_{j} ρ \frac{π}{4} {d}^{2} + {v\_tube}_{n} ρ \frac{π}{4} {D}^{2}$$

$$\dot{m} = V ρ \frac{π}{4} {D}^{2}$$

Where ρ is the air density, d is the diameter of the perforations, D is the diameter of the tube. The equations above provide the velocities inside tube (i.e. v_tube₁; v_tube₂; v_tube₃; . . . ; v_tubeₖ).

In a third step of the first iteration (see 42 in FIG. 21) the velocities inside the tube are used to calculate a set of pressures (ρ₁, ρ₂, ρ₃ . . . ρₖ) corresponding to each of the perforations as explained hereinafter. The flow axially within the interior of the tube may be modelled as inviscid flow. Bernoulli's equation may be used to provide a prediction of the pressure within the tube as a function of the velocities inside tube computed in the previous step (i.e. v_tube₁; v_tube₂; v_tube₃; . . . ; v_tubeₖ). It is assumed that the velocity in the tube near the end cap is zero and the velocity at the source is V and the constant air density is ρ. The pressure at the end of the tube farthest from the source is calculated as:

$$P = \frac{1}{2} ρ {V}^{2}$$

Then this value of the pressure P is used to estimate the pressures within the tube 42 at each of the many holes numbered j=1 to k as follows:

$${p}_{j} = P + \frac{1}{2} ρ {V}^{2} - \frac{1}{2} ρ {v\_tube}_{j}^{2}$$

These pressures at each hole are computed and stored in a vector (ρ₁, ρ₂, ρ₃ . . . pₖ).

In a fourth step of the first iteration (see 43 in FIG. 21), the pressures (ρ₁, ρ₂, ρ₃ . . . pₖ) are used to calculate a new estimate of the exit velocities. The flow from the interior of the tube to the exit hole may be modelled as inviscid flow. Bernoulli's equation may be used to provide a prediction of the exit velocity as follows:

$${v\_u}_{j} = \sqrt{\frac{2}{ρ} {p}_{j}}$$

One may use the relationship above k times (for each hole number from 1 to k) to calculate exit velocity estimates at each perforation or hole (i.e. v_u₁, v_u₂, v_u₃ . . . v_uₖ). The updated exit velocity estimates v_uⱼ are different from the initially assumed distribution (i.e. v₁, v₂, v₃, . . . vₖ).

By mass conservation, the sum of the exit velocities must obey the relationship

$$\dot{m} = {\sum }_{j = 1}^{k} {v}_{j} ρ \frac{π}{4} {d}^{2}$$

In a fifth step of the first iteration the exit velocity estimates calculated in the fourth step are used to calculate a set of velocities (v₂₋₁, v₂₋₂, v₂₋₃, v₂₋₄, . . . v₂₋ₖ) to be used as starting point for a second iteration. The set of velocities are calculated as follows:

$${v}_{2 - j} = {v}_{j} \cdot \frac{( V ρ π {D}^{2} / 4 )}{{\sum }_{j = 1}^{k} ( {v}_{j} ρπ {d}^{2} / 4 )}$$

The set of velocities v₂₋ⱼ preserve the proportions among the calculated exit velocities v_uⱼ but their magnitudes are adjusted to satisfy mass conservation by scaling each value. The scaling is performed by dividing each exit velocities by the sum Σⱼ₌₁^{k}(νⱼ ρπd²/4) and multiplying it by the known mass flow supply which is (V ρπD²/4).

The resulting exit velocity distribution (v₂₋₁, v₂₋₂, v₂₋₃, v₂₋₄, . . . v₂₋ₖ) is used as an updated estimate for a second iteration. The second through fifth steps (41 through 43 in FIG. 21) are repeated for the second iteration thereby obtaining a velocity distribution to be used as updated estimate for the third iteration. The process is iterated until it converges to a stable distribution of exit velocities (i.e. v_{F1}, v_{F2}, v_{F3}, v_{F4} . . . v_{Fk}). The obtained distribution of exit velocities may be approximately elliptical if the total area of perforations is not small compared to the cross sectional area of the tube.

The density of the perforations 44 is determined by making it proportional to the inverse of the exit velocities. In an exemplary embodiment the position coordinates of the k perforations along the tube is denoted as x₁, x₂, x₃, x₄, . . . xₖ where xₖ is the distance between perforation k and a reference point on the tube between the air source and the first perforation. The positions xⱼ (with j between 1 and k) may be calculated from the set of equations:

$$( {x}_{j + 1} - {x}_{j} ) = α \cdot \frac{1}{{v}_{Fj}} ; ( where 1 \leq j \leq k )$$

Where α is determined by setting the distance between the first and last perforation to the desired length: (xₖ−x₁)=L.

The above equations enable the skilled artisans to derive the mathematical expressions x₁=Φ₁(V, d, D, ρ, k, L); x₂=Φ₂(V, d, D, ρ, k, L); x₃=Φ₃(V, d, D, ρ, k, L); . . . xₖ=Φₖ(V, d, D, ρ, k, L), thereby providing the positions and density of the perforations as function of parameters (V, d, D, ρ, k, L). The functions Φn(V, d, D, ρ, k, L) may be expressed by closed form expressions.

Alternatively, the set of parameters may be associated the resulting positions, (V, d, D, ρ, k, L)→(x₁, x₂, x₃, x₄, . . . xₖ), determined by the above algorithm thereby forming the function (x₁, x₂, x₃, x₄, . . . xₖ)=Φ(V, d, D, ρ, k, L). The function Φ(V, d, D, ρ, k, L) may be expressed by a closed form expression.

The positions and density of the perforations computed in the CPU 38 is implemented by a cutting die 45 which is located at positions over the clear plastic tube according to the desired perforation positions/density (i.e. x₁, x₂, x₃, x₄, . . . xₖ). The resulting perforations distribution will essentially follow an inverse of an elliptical function. By making the density of perforations an inverse of an elliptically shaped function, the resulting air distribution within the surgical area is uniform throughout providing an advantage in quality of the surgical outcome.

In an exemplary embodiment of the invention a method for manufacturing a portable surgical system may include: (1) running on a CPU the iterative computation described above; (2) receiving, from the CPU, at a machine for cutting perforations into the tube material a set of numbers corresponding to the positions (x₁, x₂, x₃, x₄, . . . xₖ) of the perforations; (3) cutting the perforations into the tube materials at positions (x₁, x₂, x₃, x₄, . . . xₖ) received from CPU.

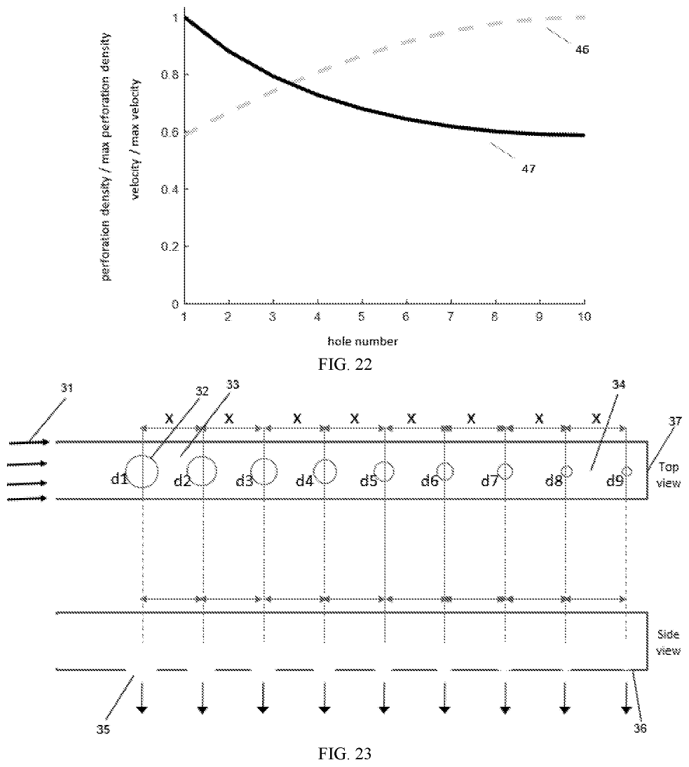

As an illustration, the resulting velocity distribution and perforation density distribution are graphically depicted in FIG. 22. This depiction is for a case with ten perforations in the collapsible flexible tube and it will be understood that the method generalizes to other numbers of perforations. The hole number is on the x axis and the exit velocity 46 and perforation densities 47 (normalized so that the maximum values are unity) are represented on the y axis.

In another exemplary embodiment the above uniform air distribution can also be achieved via an alternative configuration of the perforations in the flexible tube as shown in FIG. 23. In this configuration the perforations are equidistant (distance depicted as x in FIG. 23) while the diameter of the perforations varies (i.e. d₁, d₂, d₃, . . . dₖ) such that the air flow through each of the perforations is identical and 1/k proportion of the total flow through the manifold. The goal in such a case is to integrate the total area of perforation for each given, uniform distance xᵢ. A system of dies may be used to cut the correct perforation diameter at points x₁, x₂, x₂, . . . , xₖ.

Another alternative embodiment of the air handling system inside the enclosure instead runs airflow longitudinally caudally to cranially, along center of top.

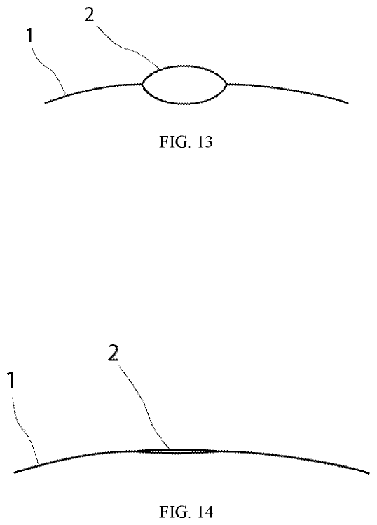

The portable surgical system may include a flexible tube 2 (as depicted in FIGS. 1, 2, 11, and 20) configured to act as a valve system, as described with respect to FIGS. 13 and 14, such as to prevent air backflow from the surgical enclosure into the fan and filter. FIGS. 13 and 14 show a cross-section through a portion of the surgical enclosure 1 and the flexible tube 2 attached to or incorporated into the surgical enclosure 1. FIG. 13 shows the flexible tube in an expanded state when air is blown from the air supply system 5 into the surgical enclosure. FIG. 13 shows the axial view with the overhead inlet tube valve in the enclosure open during active air inflow, signaling adequate flow. FIG. 14 shows the axial view with the tube valve FIG. 13 pinched closed by the enclosure's positive pressure, thus sealing the system and preventing backflow. FIG. 14 shows the flexible tube in a collapsed state when air pressure inside the enclosure is pushing the air from the enclosure towards outside the enclosure. The collapsed tube 2 prevents the air from exiting the enclosure.

The collapsible tube may be made of flexible material such as to switch from open to close state, and vice versa, based on airflow. The airflow passes from air supply system first through an inflow tube valve 2 comprising a sealed tube of collapsible plastic. When there is net positive airflow through the tube toward the manifold in this configuration, the transmural pressure is positive relative to the enclosure, and the tube is forced open. When there is no airflow or reversed airflow, the transmural pressure drops relative to the enclosure, causing longitudinal collapse of the tube. This tube valve reduces further flow in the setting of enclosure excess pressurization as the enclosure positive pressure produces transmural pressure favoring valve collapses; prevents flow reversal as enclosure positive pressure seals off air outflow through the valve; and also serves as an indicator of adequate airflow indicator by virtue of its inflation. The airflow then proceeds to a manifold 3, implemented as above in the horizontal manifold system. The relative lengths of the valve and manifold are determined by procedural needs for pressure and airflow; but the manifold should preferably extend at least the full length of the operating-section.

E. Method for Setup of Surgical Enclosure with Respect to Standard Surgical Workflow

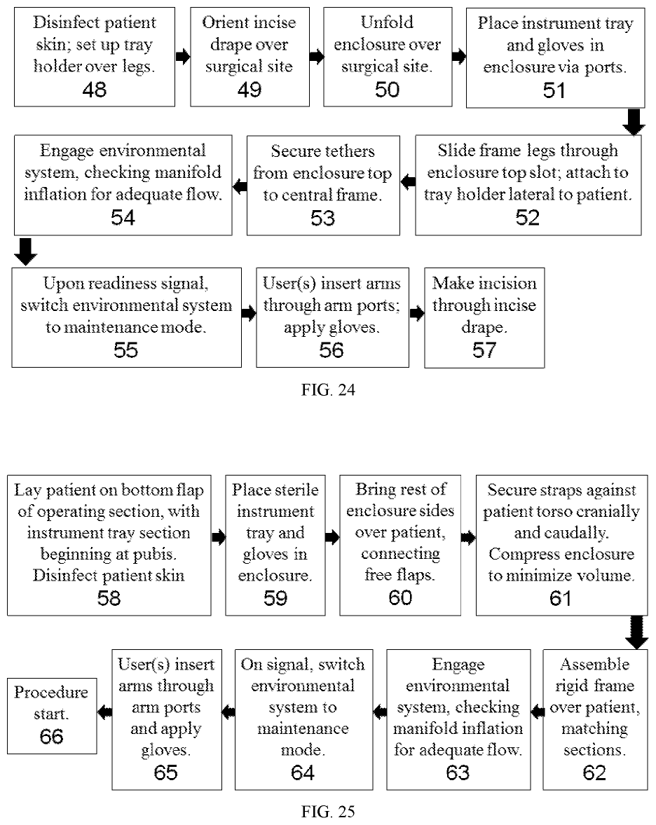

An exemplary embodiment of the present invention also discloses a method for using the ultraportable surgical system comprising the steps described in FIG. 24 flowchart. The sterile field, which corresponds to the draped areas in standard procedural setup, includes the entire enclosed area and the sleeves. This method applies for all embodiments utilizing the incise drape interface. The users first disinfect the skin 48 of the patient as per usual protocol using any of the standard skin antiseptic agents, provided they are permitted to dry fully before applying the incise drape. Users then orient 49 the enclosure with the incise drape over the planned surgical site and the instrument-section extending caudally, set up the enclosure 50, and add needed instrument tray and gloves via the material ports 51. As the entire system comes pre-sterilized in packaging, the air inside is sterile until the sterile instrument tray is placed. The enclosure is then connected to the frame 52 which in turn is stabilized on the instrument tray holder, strapped down for additional stabilization against the patient or operating table 53, and the environmental control system is turned on 54. Inlet tube valve inflation is utilized as the indicator of adequate airflow through the environmental system. The first inflation is thus also an initial purge of any contamination introduced during that step. When the system is adequately inflated, or an indicator is activated, the environmental system is switched to maintenance mode 55. At this point, users can place arms through the arm ports, apply gloves or overgloves in standard protocol 56, and initiate the procedure 57. Maintenance mode is an option for procedures in which the air changes are planned to be different than the ones used for initial inflation or that opts to recycle air through an exhaust system to prolong filter life span, but it can also be no change from prior mode. For arm port use, it is recommended that providers wear one pair of sterile undergloves, then don the second pair of gloves inside the enclosure in standard double gloving procedure to seal the sleeve port embodiments of the arm ports.

At the end of the procedure following any appropriate skin closure and dressing application, users remove the tray and any items from inside the enclosure, clear any blood or bodily fluids within the enclosure, doff gloves then remove arms from the arm ports, turn off the environmental control system, remove the air supply tubing from the air handling inlet, pull the enclosure off of the frame as well as off of the patient, and dispose of the enclosure.

For embodiment systems not utilizing incise drapes, setup methodology is described in FIG. 25. In this scenario, the user positions the patient directly over the bottom flap of the operating-section 58, places instrument and gloves in planned enclosure 60, connects the bottom flap against the side of the enclosure 60, clinch the enclosure cranially and caudally against the patient 61, then assembles the frame while connecting to the enclosure 62. The environmental control system is engaged 63 with monitoring of wind sock at air inflow to check for adequate flow. When the enclosure is adequately filled with clean air as shown by indicator (based on air changes), the environmental system is switched to maintenance mode 64. At this point, users can place arms through the arm ports, apply gloves or overgloves in standard protocol 65, and initiate the procedure 66.

Although only a few embodiments have been described in detail above, those skilled in the art can recognize that many variations from the described embodiments are possible without departing from the spirit of the invention.

F. Supporting Studies

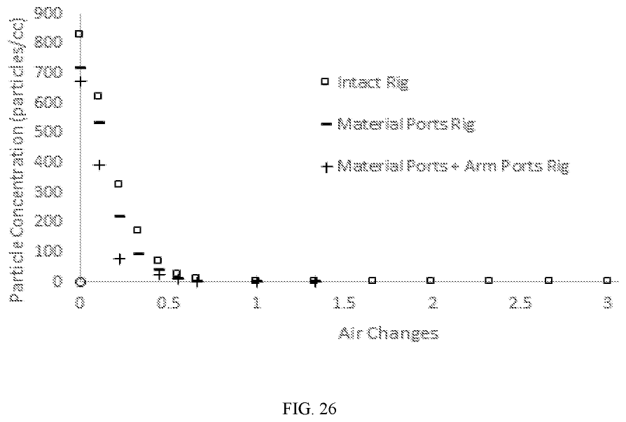

Inventors have implemented various embodiments, such as the ones described herein among others, by manufacturing and testing fully self-contained portable surgical systems. In Teodorescu et al (2016) inventors have demonstrated an early proof of concept and bench tested a functional prototype enclosure (FIG. 26). Inventors have further demonstrated that even with enclosure contamination to level found in machine shop utilizing charcoal burning, 2.25 air changes were adequate to consistently bring contaminant particulate levels to 0 particles per cubic centimeter. Subsequent systems reduced susceptibility to enclosure contamination and improved setup speeds through the protocols described above (e.g. as described in Teodorescu et al 2017).

The features of the invention disclosed herein, as specified by actual surgical end-users, distinguish it from prior art by enhancing usability, ergonomics, independence from external resources, and reliability under field conditions. The inclusion within the enclosure of only the surgical site, excluding the remainder of the patient body from the sterile field, particularly high-contaminant regions such as the oropharynx or the genitals, improves the efficacy of the system. The invention's ability to isolate the surgical wound's contaminant production, such as blood and bodily fluids, and contain these through the life cycle of the product, is also a key feature.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalent.

G. The Sterile Sleeves

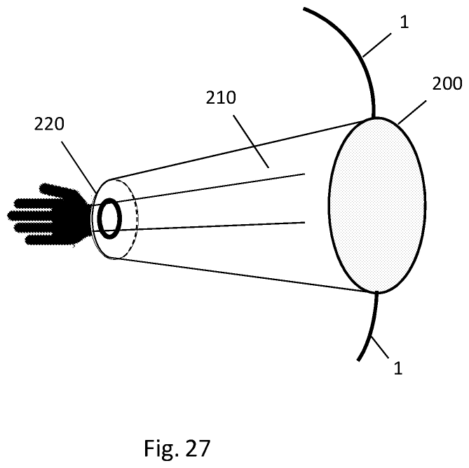

The portable surgical system may further include sleeves for accessing the inside of the enclosure. As mentioned in the section C regarding “Ports” the second form for the arm port may include sleeves such as shown in FIGS. 2, 27 and 28. In an exemplary embodiment described with reference to FIG. 27, the sleeve 210 may include a hollow cylinder or frustrated cone of material (e.g. fabric; rubber; plastic; or a composite/combination that may include fabric, plastic, surgical glove material, latex and other materials) that may taper toward the inside of the enclosure 1 away from the wall. The sleeve end may be free or may incorporate a cuff of elastic material to fit against the user wrist. In another exemplary embodiment the sleeve may end in a glove. The length of the sleeve is such as to permit ergonomic handoff of instruments among ports at contralateral ends of the system. At the other end, the sleeve is attached to the surgical enclosure 1 at 200 (e.g. by sewing or heat).

The sleeve material may include one or more layers of materials, such as, but not limited to: fabric, rubber, thermoplastics or a combination that may include fabric, plastic, surgical glove material, latex, polyurethane, polycarbonate, acetal copolymer polyoxymethlene, acetal homopolymer polyoxymethylene, acrylic, nylon, polypropylene, polystyrene, or thermoplastics that are sufficiently non-brittle to act as a cloth-like material. The layers may be configured to focus on ergonomics, ease of use, or functional properties. These layer's functional properties could include: material's ability to reduce heat retention, increase heat retention, wick up moisture, decrease friction, increase friction, or other properties that would benefit the comfort or functionality of the sleeve. For example, one or more of the layers may be made of a material such as to create a comfortable feel when touching a patient and such as to absorb/wick up moisture from inside the enclosure.

In an exemplary embodiment, the sleeve material may have the structure (and may be fabricated by the processes) described in the international patent application PCT/US19/32148 filed on May 14, 2019 and titled “Sterile Sleeves for Portable Surgical Systems” which is incorporated herein in its entirety for all purposes as if fully set forth herein.

An exemplary embodiment is described hereinafter with reference to FIG. 27. The sterile enclosure 1 of the portable surgical system includes a port 200. The end of the sleeve 210 is attached to the port 200 such as to form a substantially impermeable seal as shown in FIG. 27. The attaching of sleeve 210 to the port 200 may be performed by sewing, thermal adhesion, or other processes known to the skilled artisan. The sterile sleeve 210 and the port 200 are configured to be used by an operator to access the sterile enclosure/environment.

The sleeve may further include a mechanism 220 (FIG. 27) configured to secure the user's wrist or arm or hand in place with respect to the sleeve, such as: a strap, an elastic band, a string, or thread. The mechanism 220 may further include a strap with an adhesive, velcro, or other friction based adhesion such as those found in athletic foam wraps. The strap may be tied off in a manner similar to that of sweatpants/scrubs, or wrapped then tied like a present.

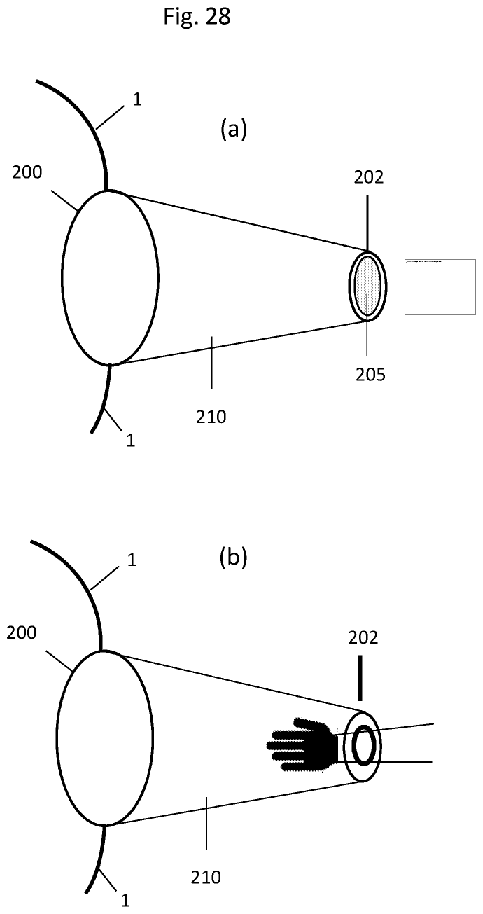

The sleeve may further include a removable sterile cover 205 (shown in FIG. 28) configured to cover the opening 202 prior to use of the sleeve. The cover 205 is configured to be removed by operator prior to accessing the port and to cover or seal the port when the port is not used. The function of cover 205 is to retain the sterility of the internal portion of the sleeve. The cover 205 may be made as a disposable perforated element that can be torn off, a reusable cover that can be adhered to the end of the sleeve after proper sterilized preparation of the sleeve, or other adhesive or covering that can act as a barrier for port 200.

A method of using the portable surgical system is described hereinafter with reference to FIG. 28. Prior to using the sleeve, the cover 205 is covering the port 200 substantially sealing the inner of the enclosure 1 from the outer environment as shown in FIG. 28. The operator proceeds to remove the sterile cover 205. After the sterile cover 205 is removed, the operator inserts her hand in the sleeve through opening 202 as shown in FIG. 28. The operator may be sterile to at least his or her wrist, prior to entering the sleeves. After the operator has placed his or her hand within the opening at 202, extra material can be tightened through an internally incorporated or external strap, elastic band, string, thread, or simply wrapped around the operator's wrist. Once secured, the operator will then invert the sleeve with their hand still secured, allowing the side of the sleeve to cover over their forelimb, thereby arriving at the configuration shown in FIG. 27. In this embodiment, the sleeve side may be long enough to reach over the elbows, shoulders, or have additional slack beyond the length of the operator's forelimb. Once fully inverted, the operator may elect to place a fitted glove on and over their hand and wrist, effectively covering the point of entry 202 and further creating a better seal around the operator's wrist.

H. The Exhaust and the Fluids and Debris Elimination System

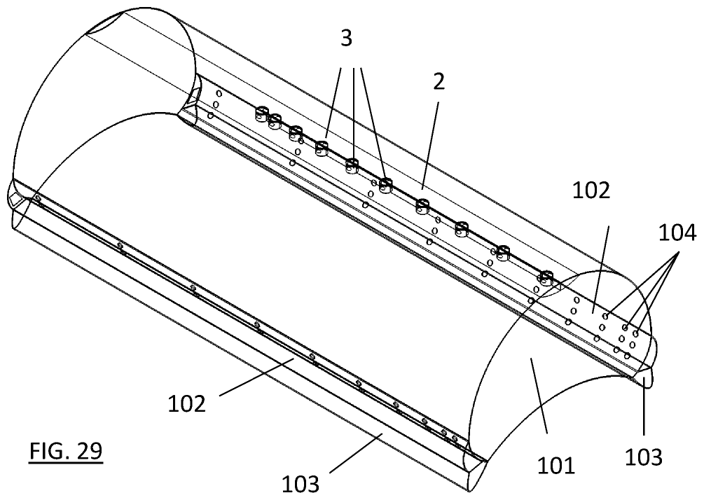

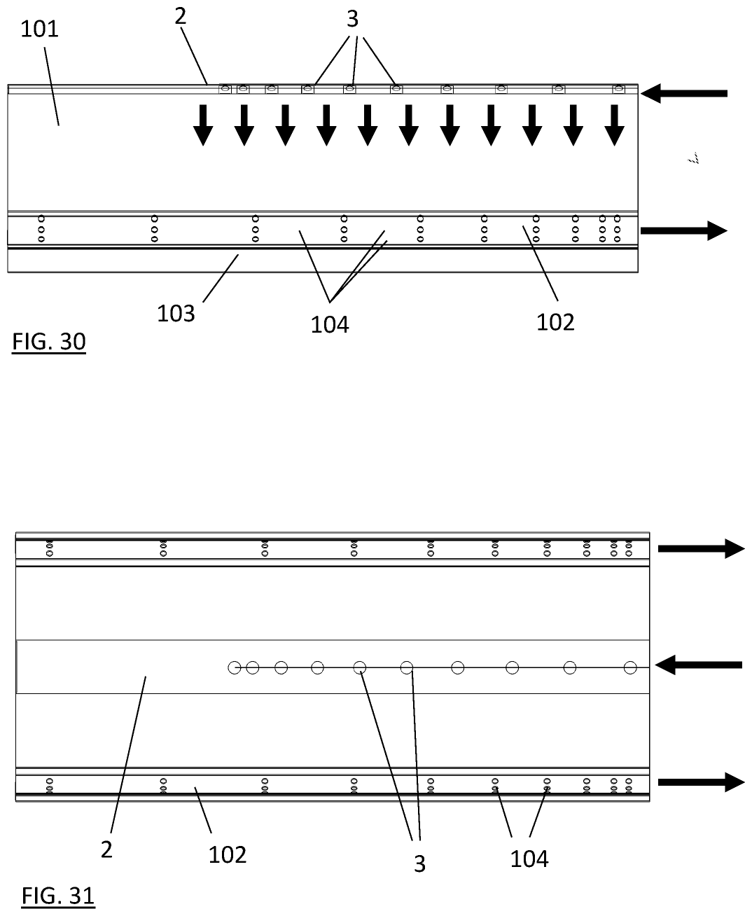

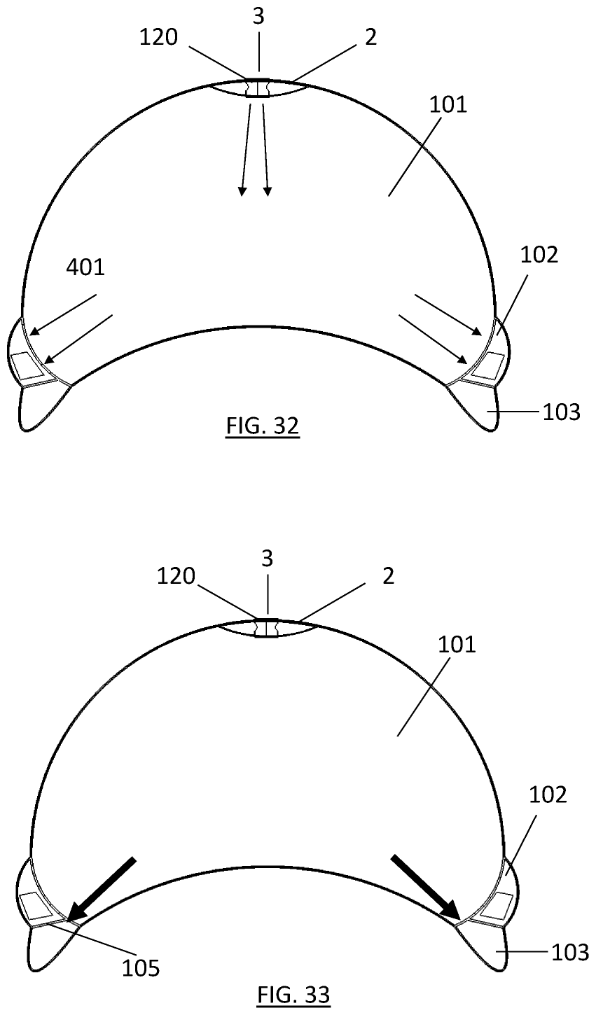

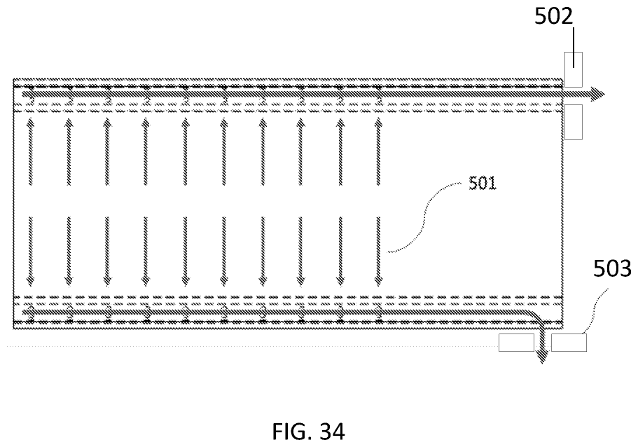

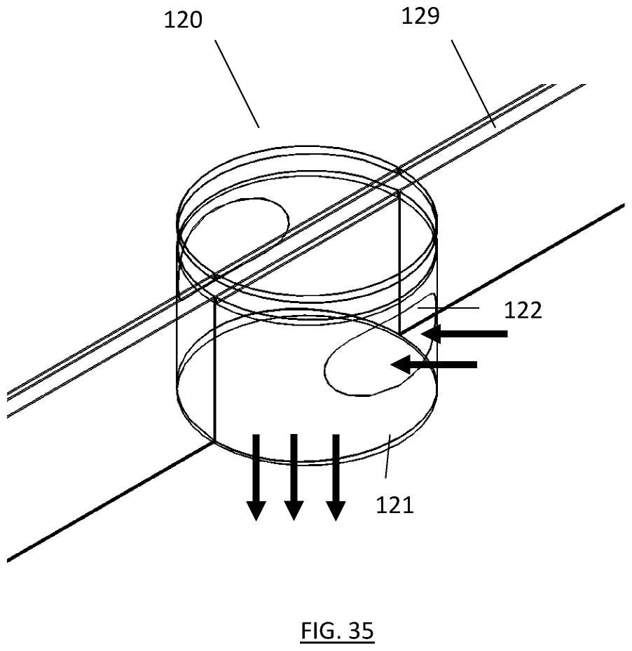

The portable surgical system may include an exhaust system and a fluids and debris elimination system which are described hereinafter with reference to FIGS. 29-34. FIG. 29 shows a schematic perspective view of a portable surgical system including an exhaust system and a fluids and debris elimination system. FIG. 30 shows a side view of the portable surgical system including an exhaust system and a fluids and debris elimination system. Airflow into the enclosure, supplied by the environmental control system, is provided via a port of the flexible tube 2 as shown by the upper arrow in FIG. 30. Airflow out of the enclosure is provided via one or more ports of the exhaust channels 2 as shown by the lower arrow in FIG. 30. FIG. 31 shows a top-down view of the portable surgical system schematically depicted in FIGS. 29 and 30. FIG. 32 shows a cross section of the portable surgical enclosure schematically depicting vertical airflow out of the flexible tube 2 and airflow from the enclosure into the exhaust channels 102. FIG. 33 shows a cross section of the portable surgical enclosure schematically depicting into the elimination of liquids and debris from the enclosure into a fluids and debris elimination channel 103. FIG. 34 schematically shows the flow of the air from the enclosure into the exhaust channels and out of the exhaust channels via a set of ports. FIG. 35 schematically shows a part of an airflow guiding system which may be disposed inside the airflow supply flexible tube.

(a). The Exhaust System

The exhaust system may include a set of exhaust channels 102 (as shown in FIGS. 29-34) which may be set to run along the length and/or at the periphery of the enclosure 1 of the portable surgical system. The channels 102 may include a plurality of perforation allowing air and gases from inside the enclosure 101 to flow into the channels 102 and from there to exit the enclosure via one or more channel outlets. Peripheral placement of channels 102 may ensure that the exhaust system is disposed away from the operating areas within the center location of the enclosure 1 clean environment. The length of channels 102 may be contained within the length of the enclosure 1 or may be chosen to extend beyond the length of the enclosure 1. The length of channels 102 may be set such as to increase convenience or functionality. The length, size and cross-section of channels 102 may be selected such as to obtain the desired fluid flow, to save material, or to increase usability of the environment. An increased length of 102 may be chosen so as to ensure a longer path for fluid flow which may allow for contaminants to fall out of the fluid stream. Differing cross-sectional areas of channels 102 may be chosen to affect fluid flow, resistance to flow, fluid pressures, or material usage for reasons such as the aforementioned.

The channel outlets may include active components such as electromagnetically or mechanically controlled valves, filtering mechanisms, gates, or other devices designed to control fluid flow.

In an exemplary embodiment, the channels 102 may include a plurality of holes 104 (as shown by FIGS. 29-34) allowing air and gases from inside the enclosure 101 to flow into the channels 102 and from there to exit the enclosure via one or more outlets. The size of the holes 104 may be chosen such as to prevent the efflux or influx of certain particles or debris. The channels 102 may include a sufficient number of holes 104 such as to provide sufficient efflux of fluid or related substances into channel 102. The holes 104 may be disposed along the length of 102.

In some exemplary embodiments, the holes 104 may be disposed at locations where they will be in direct proximity to a region of interest within the enclosure 1. The region of interest may be the surgical operating area within the enclosure 1. In some exemplary embodiments, the holes 104 may be disposed at locations where they will be away from the region of interest within the enclosure 1. The holes 104 may be disposed in close proximity to the region of interest such as to remove fluids surrounding the region of interest, to increase fluid flow over the region of interest, or to direct the fluid flow in a desired direction or velocity. The holes may be disposed to be away from the region of interest such as to decrease fluid flow over the region of interest or to direct the fluid flow in a desired direction or velocity. In other embodiments, the holes 104 may be designed to have different diameters, resistance to flow, or varying spacing such as to more finely tune the flow entering the exhaust channel 102. In an exemplary embodiment, the system is designed to maintain substantially constant efflux.

The total cross-sectional area of holes 104 (i.e. the sum of the cross-sections of all holes 104) may be chosen to be greater than the total cross-sectional area of the channel outlets (such as the outlets 502 or 503 in FIG. 34) with the purpose of maintaining a positive pressure gradient within both the enclosure 1 and the channels 102. This will allow for a completely passive system to exhaust while also maintaining positive pressure within the environment. Additionally, the channel outlets, such as 502 or 503 in FIG. 34, may be designed to have active components such as electromagnetically or mechanically controlled valves, filtering mechanisms, gates, or other devices designed to control fluid flow.

The exhaust channel 102 may be configured to exhaust the airflow 401 (see FIG. 32) such that the airflow from the surgical site minimizes its recirculation within the environment and directs an acceptable amount of flow away from the surgical site.

An essentially uniform airflow (as shown by 501 in FIG. 34) from the enclosure into the exhaust channels 102 may be accomplished by varying the density or the size of the holes 104 in the channels. The density of holes 104 may be higher at one end of the channel, the density of holes 104 may decrease along the channel until reaching the lowest density at the other end, as shown in FIGS. 29, 30, and 31. The distribution and size of the holes may be configured such as to obtain an essentially uniform airflow into the enclosure. In an exemplary embodiment the holes 104 density along the channels 102 decreases according to the inverse of an elliptically shaped function. Such holes distribution is known to lead to an essentially uniform airflow into the enclosure (as shown by 501 in FIG. 34).

In an exemplary embodiment of the invention, an exhaust channels 102 (as shown by FIGS. 29-31) may include a plurality of perforations disposed at such positions (x₁, x₂, x₃, x₄, . . . xₖ) along the channel as to create uniform air flow. The exemplary embodiment in FIGS. 29-34 illustrates a channel 102 including a plurality of perforations disposed on axial rows along the channel. The channel may include one or more axial rows of holes disposed on the channel such as to cover the channel's surface area interfacing the enclosure. The multiple axial rows may be essentially parallel with each other and with the axis of the channel.

The perforations are disposed along the channel such that the axial positions of the perforations along the flexible tube may follow a mathematical relation (x₁, x₂, x₃, x₄, . . . xₖ)=Φ(V, d, S, ρ, k, L), where V is the desired airflow velocity exiting the channel through the outlets, S is the cross-section area of the channel, d is the diameter of the perforations, and ρ is an air density, L is the length of the perforated section, and k the number of perforations in a row. The mathematical relation Φ(V, d, S, ρ, k, L) is determined via the same consideration as the ones explained with reference to the perforations in the flexible tube see e.g. FIG. 20 described in the section D of this application (titled Environmental Control System).

In an exemplary embodiment, an exhaust channel 102 further includes a channel outlet 502 which is in line with the channel 102 and with the airflow through the channel 102 (as shown in FIG. 34). In an exemplary embodiment, an exhaust channel 102 further includes a channel outlet 503 which is perpendicular on the channel 102 and on the airflow through the channel 102 (as shown in FIG. 34). In an exemplary embodiment, the exhaust system includes both an outlet such as 502 and an outlet such as 503. The cross-section size of the exhaust outlets 502 and 503 may be selected such as to obtain the desired airflow and/or pressure inside enclosure 101. The outlets at 502 or 503 may include valve structures, selectively permeable membranes, permeable membranes, filtration mediums, no material, or material that otherwise selectively allows for the exit of fluids or its contaminants. The outlets may be configured to prevent the influx or efflux of fluids or contaminants.

The exhaust system may be configured such as to cause an efflux of fluid flow 501. The exhaust system may be configured such as to essentially prevent the influx of fluid, particulates and undesirable substances into the enclosure from the outer environment via the outlets. The exhaust system may be configured such that the positive pressure, coupled with the direction of flow changes, as well as with the particular geometries of the exhaust, essentially prevent the influx of fluid, particulates and undesirable substances into the enclosure from the outer environment via the outlets 502 or 503. The outlets 502 or 503 may be disposed in line with the fluid flow or orthogonal to the fluid flow in order to resist or permit fluid flow, increase or decrease static or dynamic fluid pressures, change direction of the exhausted fluids, improve user convenience, or to further resist the influx of undesired substances.

(b). The Debris and Fluids Elimination

In an exemplary embodiment, the debris and fluids elimination system includes one or more debris elimination channels 103 as shown in FIGS. 29-33. The channels 103 may be disposed under the exhaust channel 102 and may be configured to collect blood, water, bodily fluids and other substances which need to be eliminated from the inside of the surgical enclosure. The channels 103 may be configured to eliminate blood, water, bodily fluids and other substances out of the enclosure and into a collection reservoir. The collection reservoir may be configured to collect and store biohazardous materials such as blood and bodily fluids. The reservoirs may be attachable to outlets of the channels 103, may be disposed outside the enclosure, and may be removable from said outlets such as to allow safe disposal of the materials collected in the reservoir.

The channels 103 may include a set of openings or perforations allowing blood, debris, bodily fluid, and other substances accumulated in the enclosure to enter into the channels 103. The fluid and debris elimination channels 103 may be disposed in the lower part of the enclosure (see e.g. FIGS. 32 and 33) and may be configured such that blood, debris, bodily fluid, and other substances accumulated in the enclosure are moved into channels 103 via gravity, airflow and other forces as shown by the arrows in FIG. 33. The channels 103 may be configured such that blood, debris, bodily fluid, and other substances accumulated into channels 103 are moved into one or more collection reservoirs. The fluid elimination channels 103 and the airflow exhaust channel 102 may be adjacent to each other and separated by a wall 105 such that there is no possible contamination of the exhaust channel 102 with fluid retained in channel 103.

I. Redirecting Flow in the Flexible Tube

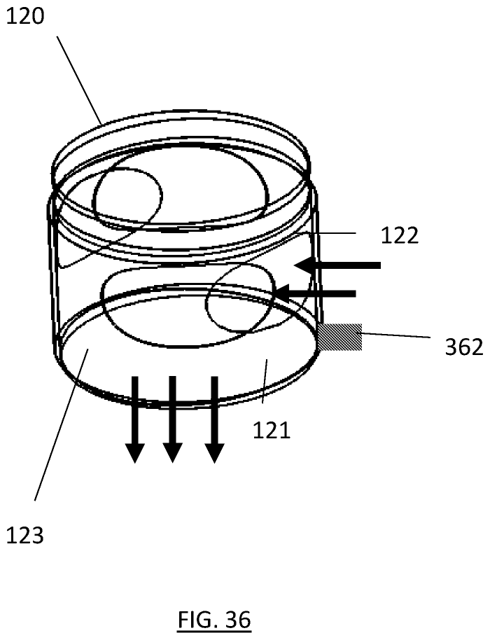

The flexible tube 2 of the environmental control system (described in this application at section D “Environmental Control System”) may be disposed with respect to channels 102 and 103 as shown in FIGS. 29-33. The flexible tube 2 may include perforations 3 as described in section D “Environmental Control System”. The flexible tube 2 may include a flow guiding system configured to guide airflow through the flexible tube 2 and out of the tube 2 into the surgical enclosure. The flow guiding system may include a central divider 129 and a plurality flow-guides 120 (as shown in FIGS. 29-33 and 35). Each of the flow-guides 120 may be disposed on top of a perforation 3 and may be essentially co-axial with the perforation or desired direction of flow. A flow guide 120 may be disposed on top of perforation 3 in a direction that is co-axial to the direction vector that is desired by the user. A flow guide 120 may be a component made of plastic or any other suitable material having a cylindrical or curved shape and may include side openings 122 and a base opening 121 (as shown in FIG. 35). The side openings 122 may face towards the inner of the flexible tube 2 whereas the openings 121 may face towards the surgical enclosure. The flow guide 120 will enable air from the flexible tube 2 to be guided into the surgical enclosure via openings 122 and 121 as shown by the arrows in FIG. 35. Other designs may include geometries that are more linear or planar such as referenced in FIG. 36 for ease of manufacturing or for altering the shape, velocity, or volume of the output flow. The goal of the flow guides is to interrupt the forward momentum of flow and redirect it along a different vector as configured by the flow guide. For instance, a flow guide may be angled at a certain angle (e.g. 45°) slope with the intention to direct the flow along an angled trajectory instead of a trajectory normal to the exit of the flow guide 120. The center divider 129 is configured to prevent flow from one side to the other of the flexible tube thereby limiting vortexing. The inventors herein have performed computer simulations showing that flow guiding systems such as described above lead to substantially uniform and laminar airflow from the flexible tube into the surgical enclosure.

A flow guide 120 may further include a mini-valve 130 for adjusting the airflow through the flow guide 120. In one embodiment the mini-valve 130 may include a cylindrical surface gliding over the openings 122 such as to adjust the opening size (as shown in FIG. 36). The magnitude of the opening may be adjusted manually or via electrical actuators. The flow-guide may further include a mechanism for adjusting the direction of the airflow into the surgical enclosure (e.g. an angle of the airflow with respect to an axial direction of the flow guide). The magnitude and directions of the airflow through flow guides may be adjusted via actuators (or other components such as motors, servos, piezo electronics, springs, memory alloys, or any combination of the above) controlled by a computer connected with environmental control system. The computer may include an application or software for automatically adjusting the airflow magnitude and direction through the flow guides such as to obtained the desired environmental parameters (e.g. pressures, airflows, etc.) inside the enclosure.

In an exemplary embodiment a flow guide 120 may include an outer cylinder 123 concentric with an the cylinder with perforations not too dissimilar to perforation 122. The perforation of the outer cylinder 123 may be configured to match in size with 122. If varied amounts of airflow are required, the outer concentric cylinder can be rotated to expose differing levels of overlapping perforations 122 and 123, effectively creating controlled volumes of airflow that are correlated to the amount of area of overlap. A higher level of perforation overlap of 122 and 123 would allow for more unobstructed airflow, whereas a lower levels of perforation overlap would result in a decreased airflow. The outer concentric cylinder could rotate via manual mechanical means with the user rotating and adjusting the level of overlap. Also, the level of overlap between 122 and 123 may be controlled by the environmental control unit 4. Such control may be permitted using actuators, motors, servos, piezo electronics, springs, memory alloys, or any combination of the above.

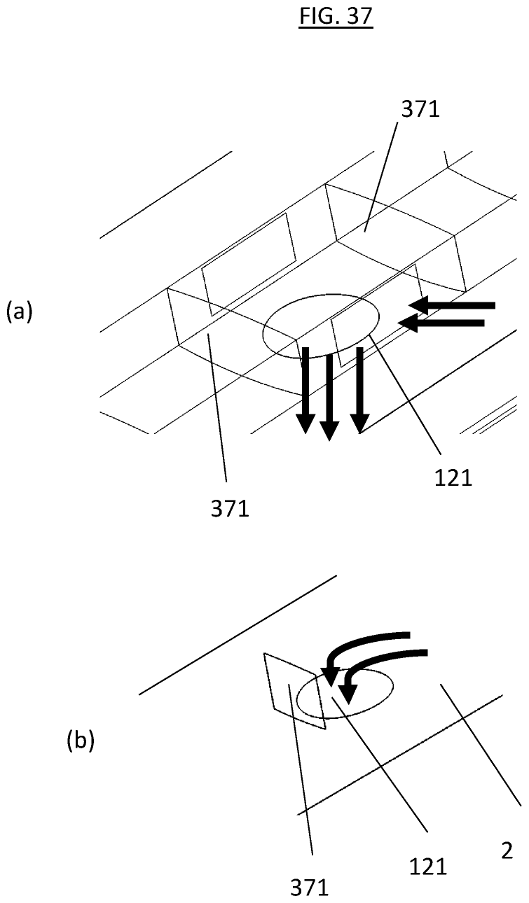

In another embodiment, the flow guiding system may direct airflow downwards from tube 2 with static openings and barriers 371 to the direction of flow in tube 2 which direct airflow through exit hole 121, as exemplified in FIG. 37 (a) and (b).

The shape, size, and orientation of a flow guiding system highly affects airflow. The flow guides may include a system configured to dynamically change the geometric properties of elements of the flow guiding system and the flexible tube 2. The flow guiding system may include materials that are deformable and which may be used in, around, or otherwise augmenting the structure of a flow guiding system or flexible tube 2. Materials that may be used include ductile metals, shape memory alloys, piezoelectric materials, photovoltaic materials, electroactive polymers, magnetorestrictive materials, magnetic shape memory alloys, smart inorganic polymers, pH-sensitive polymers, temperature-responsive polymers, halochromic materials, chromogenic systems, ferrofluids, photomechanical materials, polycaprolactone, self-heating materials, dielectric elastomers, magnetocaloric materials, thermoelectric materials, chemo responsive materials, or other materials that may deform through physical means such as bending, torsion, compressing, shearing, tension, or fatigue.

In a preferred embodiment, a flow guide 120 may include a memory shape alloy, for example Nitinol, lining the perimeter of perforations 122, allowing for the constriction or dilation of the perforations 122 when an electric current is passed through the alloy. This will serve to decrease airflow during constriction or increase airflow during dilation. Similarly the perforations included in airflow perforations 104, the bottom of the flow guide 120, the exit hole of 102, or augment exits 502 and 503 may include components made of memory shape alloys. For each of these perforations, they can be similarly controlled through introduced electrical current to allow for the deformation of the perforations as described above. In another embodiment, another such material that may be used is a temperature responsive polymer, whereby each of the perforations, openings and holes described above may be covered or lined with the temperature responsive polymer. Increasing airflow through openings may be achieved increasing the released heat, since introducing heat to the temperature responsive polymer causes it to deform and to dilate the openings. Many of the above materials may be used in similar manners such that when subjected to their responsive stimulus would cause them to act in similar ways. As described above, these stimuli may be introduced either directly from user intervention or through means of control from the environmental control unit.

The flow guides 120 and flexible tube 2 may also be geometrically modified or influenced using dynamic structures. Such dynamic structures reference geometries that are not reliant on solely material properties for changing geometries, but instead the particular means of constructing with a particular material that yields a structure that can affect the geometry of the flow guides 120 and flexible tube 2. Such structures may include inflatable air bladders that restrict, dilate, reorient, or otherwise modify the geometries of the flow guides 120 and the flexible tube 2. Such structures may exist in or around the flow guides 120 and flexible tube 2. Inflation methods may utilize a concurrent source of fluid flow from the environmental control unit or external solutions. Some external inflation modes may include compressed gas, a user blowing into the compartment, heating a material for expansion, or other reaction that causes material expansion or contraction.

In an exemplary embodiment, the walls within flow guide 120 and divider 129 may be two layered to allow for the entrapment of air. Using a separate channel, the environmental control unit may control the amount of air introduced into the compartment within flow guide 120 and dividers 129. Inflating structures 120 and 129 causes the expansion of the structures which causes the constriction of openings 122 and 121 and also decreases the volume within flexible tube 2, thereby resulting in an increased downstream resistance to airflow and causing an overall reduction in flow. The air source for the inflating structures may the same as for the surgical enclosure 1 or may be a separate air source.

In another embodiment, the flow guiding system and the flow guides 120 may include layers of material made of a memory shape alloy, such as Nitinol, wherein the layers are lining perforations 122. When an electrical current is introduced to the memory shape alloy, the layer of material will peel and curve away from the perforation 122 such that there is no longer any material preventing the inflow of air through flow guide 120. This dynamic structure may include multiple instances of this memory shape alloy such that differing levels of occlusion can be obtained. For instance, utilizing the passive shear strength of a Nitinol thread or rod, depending on the diameter, the Nitinol thread or rod will resist deflection. With this resistance to deflection, when multiple elements of Nitinol are included in the layer of material that occludes perforations 122, a differing number of Nitinol elements can be activated through electrical current. The deflection of any number of Nitinol elements would sum together and work against the summed strength of Nitinol elements that are not active, allowing the ratio of activation of nitinol elements to correlate to the level of deflection away from the perforation 122 and thereby allowing for finer control of level of obfuscation of the airway. This results in the ability to control the amount of airflow that passes through flow guide 120. In this embodiment, the nitinol activation could be either manually controlled by the user or controlled through the environmental control unit in any of the aforementioned described methods of control.

All of the above means for modifying geometries may also be used in reference to exhaust channels 102 and fluids elimination channels 103 to alter either exit fluid flow or the collection of waste products. These modifications can be made via physical user interaction with the surgical enclosure or via the environmental control unit. Any of the aforementioned environmental factors that are monitored by the sensors (e.g. pressures/airflows inside the enclosures, flexible tube and channels) as received and configured on the environmental control unit can be used as feedback to determine how the geometries of the components 120, 2, 102, and 103 should be altered to maintain an ideal steady state.

J. Monitoring and Controlling the Environment Inside the Surgical Enclosure

The surgical system may include a plurality of sensors for measuring the environmental conditions inside the surgical enclosure, outside the enclosure, the exhaust channels, the airflow supply tubes and channels and the fluids elimination channels. The sensors may include pressure sensors, airflow sensors, humidity sensors, and temperature sensors. The sensors may measure a plurality of environmental parameters such as: pressures at various locations of the enclosure, inside flexible tube 2, and in the channels 102 and 103; air-flow at various locations of the enclosure, inside the flexible tube 2, and in the channels 102 and 103; humidity; temperatures; and other environmental parameters as needed.

Information obtained from the sensors may be supplied to a computer system controlling the environmental control unit, valves and filters disposed on the channel outlets, and other controls. In response to the information received from the sensors, the computer system is configured to control the environmental control unit so as to adjust the airflow, temperature and humidity inside the surgical enclosure to the desired environmental parameters. In response to the information received from the sensors, the computer system is configured to control the valves and filters at the channel outlets such as to adjust the airflow and to eliminate out of the channels undesirable fluids and debris.

Additionally, the environmental control unit may be controlled in combination with user definable settings. These settings can be determined by continuous or discrete user definable set-points, or user selectable states. User definable states may include on, off, inflation, maintenance flow, high pressure, low pressure, dehumidify, cool, heat, brighten, or any other state that is intended to affect the measure of which is measured by one of the aforementioned sensors. By providing these set-points or states, the environmental control unit will be provided with user preferences that can be achieved by the control algorithm encoded in the computer system.

Flow may also be controlled via information received from flow sensors that correlate to laminar or turbulent flow. This information would provide the means for mechanically altering the output flow characteristics by modulating fan speed, fan blade pitch, fan blade shape, airfoil pitch, airfoil shape, baffle shape, baffle pitch, baffle permeability, aperture diameter, distance of fan to outlet, orientation of fan to outlet, shape of the outlet, or any combination of the aforementioned. By modulating these mechanical factors an effect on the flow downstream will be observed by the sensors so that the desired flow characteristics can be controlled for.

The environmental conditions inside the surgical enclosure 1 (e.g. pressures and airflow) may be adjusted to the desired state by modulating the sizes of holes 122, 121, 104, 502, 503, the diameters of 2 and 301, one or more of the geometry modifying mechanisms described above. For example, by decreasing the diameters of 104 and or 502 and 503, backpressure can build up inside the enclosure allowing for an increase of pressure within the main enclosure compartment 101, more rigidity in the walls of 1, and higher flow recirculation within the main compartment. The opposite would occur if diameters of 104 and/or 502 and 503 are increased. By increasing the size of the holes 121, 122, an increase of flow into the main chamber 101 will occur. If the holes 104, 502, and/or 503 remain the same, the effect would be similar to decreasing the diameters of 104, 502, and/or 503. The overall pressure within the compartment 101 is a result of the ratio of overall area shared across holes 122 and 121 with respect to holes 104, 502, and 503. If holes 104 are reduced in diameter but maintains an overall open area that is greater than 502 and 503, the velocity of the flow is increased when exiting the main chamber 101 and entering exhaust chamber 102. This state is useful if certain larger particulates need to be evacuated from the inside of main chamber 101. The opposite would occur if the diameters of 104 increase.

Reducing the velocity of airflow out of main chamber 101 into exhaust chamber 102 serves to prevent the evacuation of particles or elements that may need to remain inside main chamber 101. The ratio of openings 502 and 503 with respect openings 121 and 122 must remain such that the total area of the openings 121 and 122 is larger than the area of the openings 502 and 503 to maintain a pressurized main chamber 101. If it is desired to deflate the main chamber 101, a change in this ratio to have the area of the openings 502 and 503 be greater than openings 121 and 122 will yield this result. If the ratio of the area of the openings 121 and 122 remain larger than the area of the openings of 502 and 503 and the openings 121 and 122 decrease in area, this will achieve a higher pressure within flexible tube 2 and air inlet tube 301. This may be desired if the airflow is too high within the main chamber 101, or if the air pressure within flexible tube 2 or air inlet 301 is required to increase in order to maintain structural rigidity or to prevent occlusion from lack of internal pressure. This would provide a means to maintain the same amount of internal pressure in main chamber 101 while still increasing the internal pressures found within flexible tube 2 and air inlet 301.

In an exemplary embodiment, a flow guide 120 may further include a valve 123 for adjusting the airflow through the flow guide 120. In one embodiment the valve 123 may include a cylindrical surface gliding over the openings 122 such as to adjust the opening size (as shown in FIG. 36) through a mechanical, thermal, or electromagnetic actuation mechanism. In another embodiment, the magnitude of the opening may be adjusted manually and fixed into position, or in an alternate embodiment dynamically adjusted via electromechanical actuators 362. The flow-guide may further include a mechanism for adjusting the direction of the airflow into the surgical enclosure (e.g. an angle of the airflow with respect to an axial direction of the flow guide). The magnitude and directions of the airflow through flow guides may be adjusted via actuators controlled by a computer connected with environmental control system 4. The computer may include an application or software for automatically adjusting the airflow magnitude and direction through the flow guides such as to obtained the desired environmental parameters (e.g. pressures, airflows, etc.) inside the enclosure.

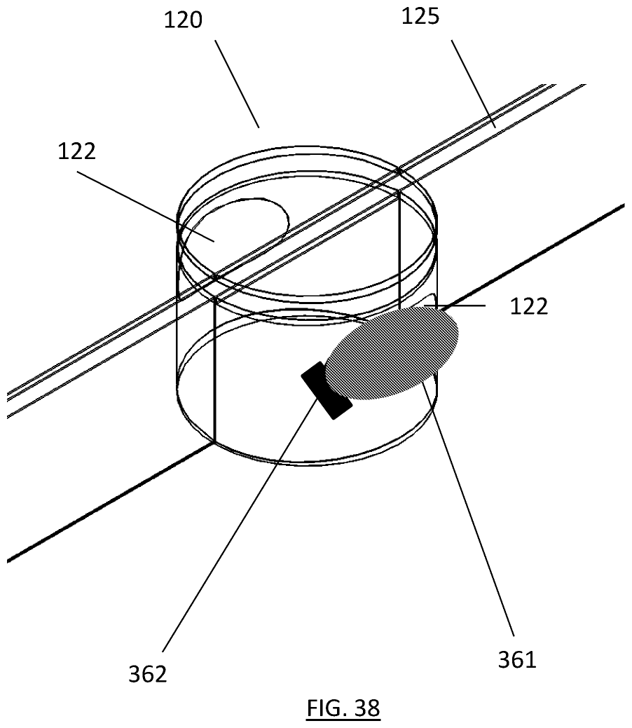

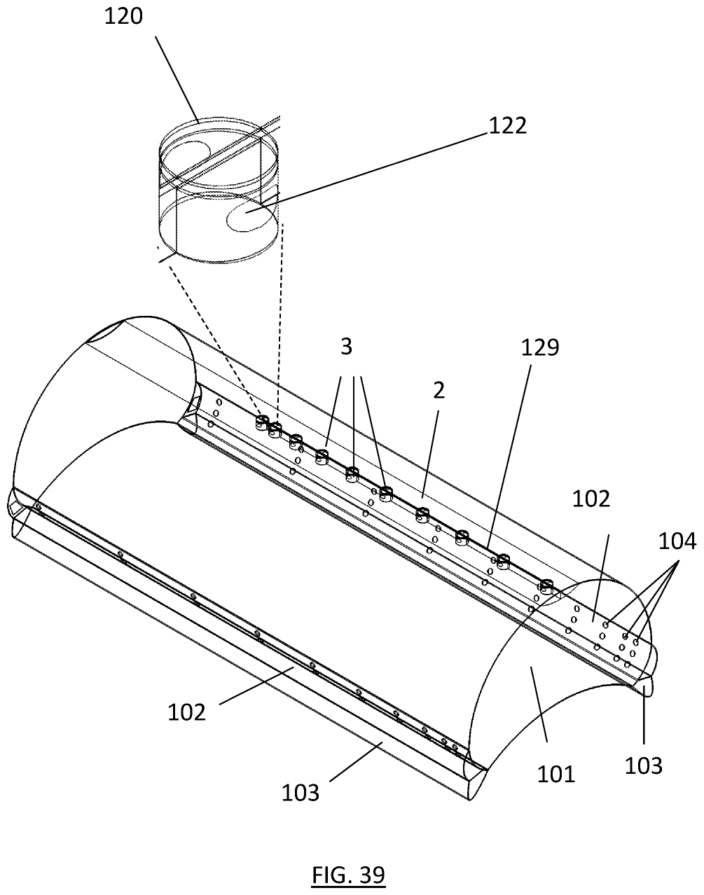

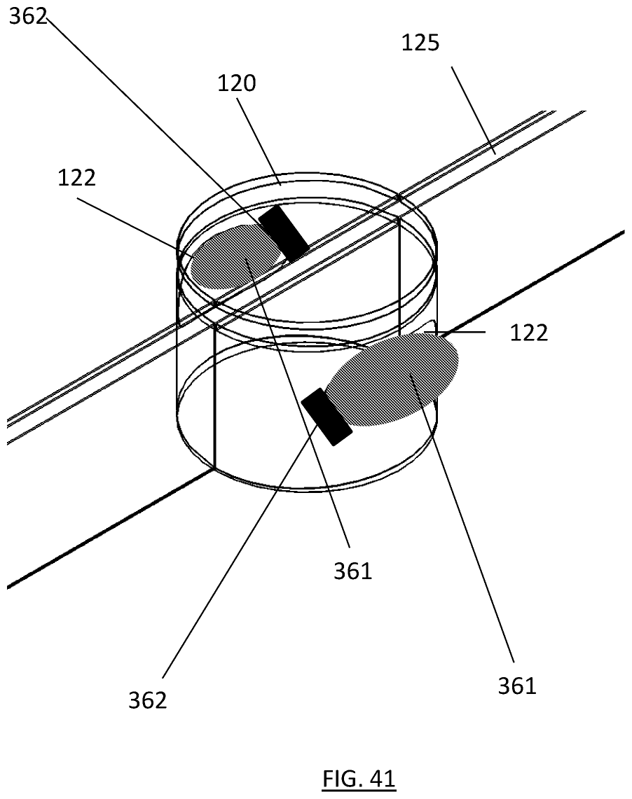

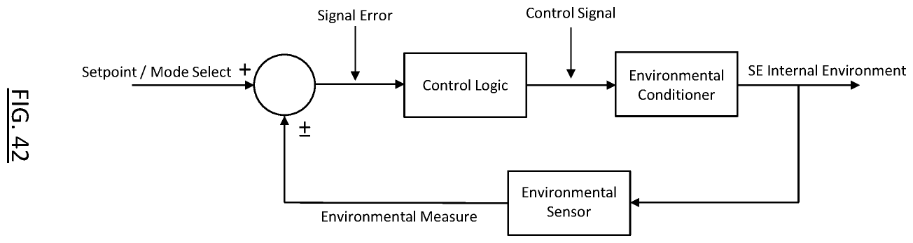

In one embodiment, each of the flow guides 120 is paired with a valve which is actuated by means of a control such as actuators connected to one or a plurality of sensors 364 inside the enclosure 1 by means of one or several cable(s) 363. FIG. 38 shows an exemplary embodiment of a valve and an actuator. FIG. 39 shows the position of the flow guide in the inflation flexible tube 2 via an exploded view. FIG. 41 shows an embodiment of the flow guide comprising symmetrically placed actuators 362 and valve flaps 361 covering individual air holes 122 in the directional flow guide element 120. These individual actuators can be moderated through a processed input from one or multiple sensors 364 disposed inside the enclosure. The sensors 364 inside the enclosure may monitor airflow, temperature, pressure, movement, location of surgical tools, patient vitals, bleeding, as well as movements of the surgeon and/or surgical tools inside the enclosure, as previously disclosed in international patent application PCT/US2019/051502 filed on Sep. 17, 2019 which is incorporated hereinafter in its entirety as if fully set forth herein. In an exemplary embodiment, the connection 363 contains multiple flexible tubes 365 each corresponding to a sensor inside a control unit 366; these flexible tubes enable pressure readings at different points inside the enclosed space.

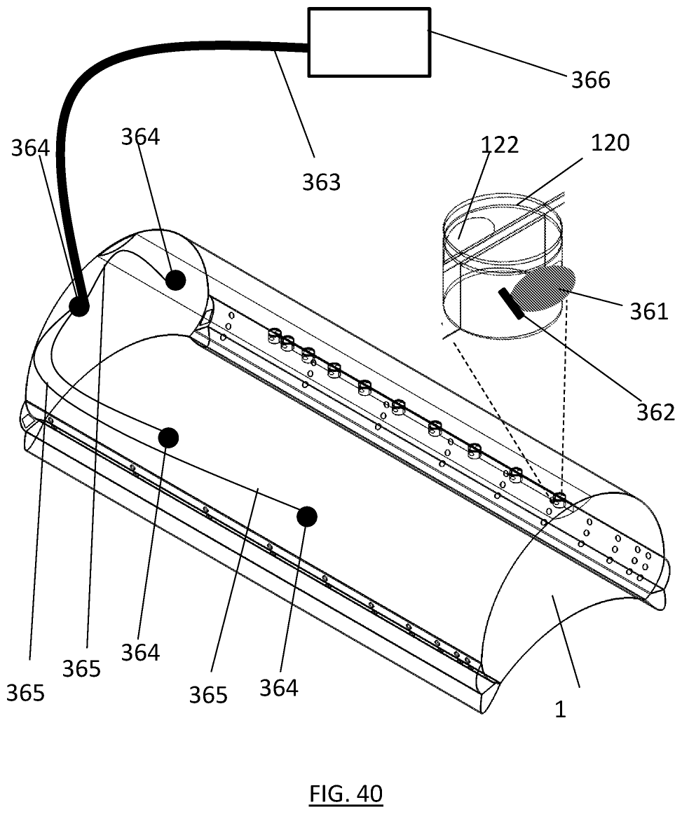

In one embodiment, the sensors can be part of the enclosure wall and connect to a control unit 366 wirelessly. In another embodiment, the sensors 364 in the enclosure 1 sidewall may connect through wires replacing the flexible tubes 363 to the control unit 366 as in FIG. 40. The control unit 366 processes the data from the sensors and actuates individual valves in airflow flexible tube component 2 and/or in the exhaust channels 102. The control unit 366 moderates the amount of airflow through each airflow valve 123 to dynamically adjust to perturbations of the airflow inside the enclosure, due, for example, surgeon hand movements, or patient breathing, and readjust the airflow profile dynamically by individually moderating the amount of airflow through openings 122 of each individual directional flow guide element 120.

In an exemplary embodiment, the control unit 366 is not hardware connected directly to the surgical enclosure sensors yet is instead an application receiving and sending data to the surgical enclosure, which may be run on separate hardware remotely, such as a portable computer, mobile phone, tablet, desktop computer, or server. In an alternate embodiment, the control unit 366 is not connected by means of a wire to sensors in the enclosure or to valves in the enclosure and instead includes its own microprocessor and wireless antenna to process information from sensors within the enclosure 1 and transmit inputs to control individual valves 123. The information from sensors as well as the state of individual control valves 123 may be shared with and processed remotely by a computer offsite, or with a mobile application running on a portable computer such as a tablet, mobile phone, or laptop computer.

The control unit 366 or a computer program functioning as a control unit for the valves 123 of the surgical enclosure may, in one embodiment, comprise predictive algorithms known to those skilled in the art such as a convolutional neural network, generative adversarial network, support vector machine, similarity learning, decision trees, or a combination of supervised and unsupervised algorithms in order to optimize airflow based on perceived visual signals, temperature signals, and/or pressure signals within the surgical enclosure 1. The surgical enclosure may relay this information to a server through a network connection, which server may then aggregate information from past surgeries and calculate an optimal airflow model which will be transmitted back to the enclosure. In another embodiment, the enclosure may run a control algorithm on site, through a connected hardware control unit 366 running an airflow control algorithm, algorithm which may be updated remotely with the latest trained model from a server aggregating data from prior surgeries from other surgical enclosures. In an embodiment, the surgical enclosure is single use, whereas the control unit 366 is of multiple uses, and may be assigned to one or several surgeons as the user. The dynamic adjustments of valves 123 within the surgical site may be performed either using a training set specific to the user or using aggregated data from many users stored on a server.

In another embodiment, the control unit 366 does not contain an active learning algorithm and instead optimizes flow using a PLC, or one or several PID controllers receiving signals from pressure sensors within the enclosure corresponding to individual air streams, or a combination thereof. In another embodiment, valves 361 are actuated through an iris electromechanical mechanism.

In another embodiment, the variable amount of airflow through valves 123 (via openings 122) can be used to recreate the air velocities of equidistant different diameter fixed holes of the embodiment in FIG. 23, as different directional elements 120 may have varying degrees of opening of valves 123 (symmetric degrees in this embodiment for either side of wall 129 within the same directional element 120) such that the air velocity at the exit point of each directional element 120 is the same. This embodiment does not require a control mechanism, whereas the valves 123 can be fixed into different positions to recreate the airflow of the embodiment of FIG. 23. In an example of this embodiment, the openings 122 are fixed in symmetrical positions on either side of wall 129 as in FIG. 41.

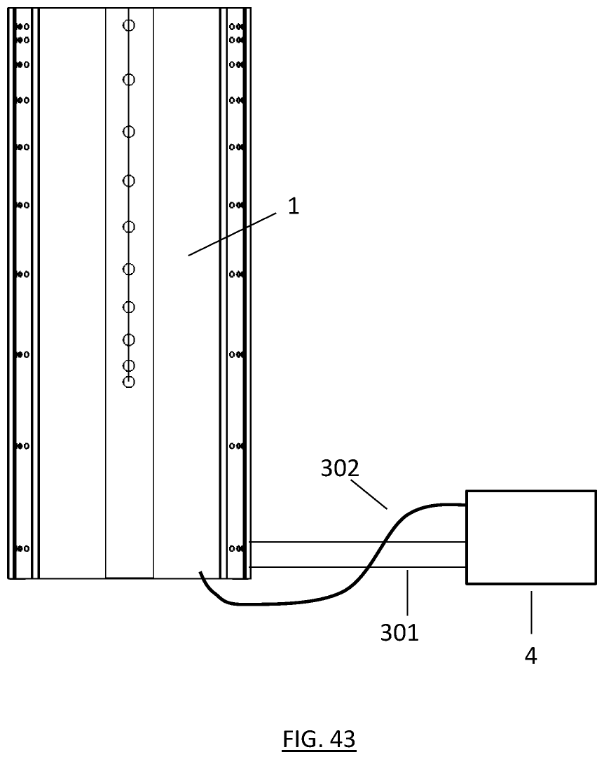

In one embodiment, the environmental control unit 4 is configured to control the valves and outlets of the surgical system in response to the environmental parameters measured by the system of sensors. The system of sensors may include a sampling tube 302 working in conjunction with one or more sensors as shown in FIG. 43. The sampling tube 302 may include an isolated channel or tube connecting the surgical enclosure 1 and the environmental control unit 4 that is separate from the airflow supply channel 301. The environmental measurements that can be collected through the sampling tube 302 are pressure, temperature, humidity, air-flow and others.

While accurate, measurements do not need to be taken through a sample tube 302 and instead may be collected from sensors either directly located on the surgical enclosure 1, leading to higher measurement accuracy, or located within environmental control unit 4, which results in less direct measurements. Both locations provide for sufficient data to allow for the environmental control unit to employ its control logic to maintain the environment within the surgical enclosure 1. By receiving data from the environmental sensors, the environmental control unit 4 can follow a similar feedback control scheme as shown in FIG. 42. The environmental control unit 4 would record the surgical enclosure 1 environment via the corresponding environmental sensor, resulting in an environmental measure that is interpreted by the environmental control unit 4. By interpreting the resulting error from the measurement, control logic can be utilized to condition the proper output to the environmental conditioner within the environmental control unit 4.

The surgical system may include environmental conditioners including one or more of the following: fans; pumps; filters; heating elements; cooling elements; humidifying elements; dehumidifying elements; elements for removing odors, particulates or microbial populations; elements that introduces odor, particulates, or microbial populations; or any combination above.

The environmental control unit may use one or more types feedback control systems including logic control systems such as those built off of relays and cam timers for ladder logic, microcontrollers, computational processing units, programmable logic controllers, programmable logic devices, complex programmable logic devices, field-programmable gate arrays, programmable integrated chipsets, systems on a chip, application specific integrated circuits, ODROIDs, application-specific standard parts, or any combination thereafter. Methods of control may include proportional control, PID control, cascade control, model predictive control, fuzzy logic. Open-loop control may be used in cases where the user or system decides that a specific control scheme is not desired and the user will instead set the environmental control unit to a user defined setting for the included environmental conditioners.

K. Dynamically Deploying Structural Supports

A portable surgical system may require additional supports to prevent collapse or occlusion of potential airflow features. Materials that maintain their shape such as spring steel, memory shape alloy, or shape memory polymers may be used in the construction of the walls, seams, or otherwise included along the structure of a feature within the surgical enclosure 1. For instance memory alloys such as Nitinol may be included in the walls and seams of the surgical enclosure 1 with the intention of keeping the same shape of the inflated structure when the structure loses internal pressure. However, since the material is a memory alloy, in this embodiment the surgical enclosure can be folded and packed away as normal. Once the surgical system needs to be unpacked, an electrical current can be introduced to the Nitinol to allow for the memory alloy to return to its stored shape and support the inflated contours of the surgical enclosure. In a similar embodiment, these memory alloys would be incorporated into the contours of the airflow channels 2, 301, 121, 122, 104, 102, 502, 503, or any combination thereof. When activated through electrical current, the memory alloy in these locations would return to the idealized inflated state to prevent any occlusion of the material caused by wrinkles or underinflation. This would aid in the prevention of blocked airflow as well as increasing the efficiency of the system, since less pressure would be needed to keep the structure inflated and thereby reducing the energy burden on the overall system. This system can also be included in the mechanical supports explained in Section C and serves as a direct improvement to the supports. A highly compact version of the supports in Section C would be made up of memory alloy that can be activated by heat or electrical current when deployed. The memory alloy will return to the same support like structure while being able to be folded into a much smaller area, allowing for the user to customize how the supports can be packed away.

The portable surgical system may be an ultraportable inflatable surgical environment that can fit in a small container (e.g., a backpack). A portable surgical system can be ultraportable, on-demand and rapidly deployable, reducing a patient's exposure to airborne particulates and a provider's exposure to patient-derived fluids. Additional features and advantages of a portable surgical system may include: allowing a user to seal sterile clear system to patient and operates via different ports; fitting into existing workflows; including integrated environmental control systems; fully self-contained; reducing scrub gear requirements; having excellent visual quality; and including reusable components. To use a portable surgical system, a provider can lay a patient on an operating table, unfold a portable enclosure of the portable surgical system, perform preoperative procedures (e.g., skin disinfecting procedure), and place the portable enclosure on top of the patient so that a drape is attached to a surgical site of the patient.

The following publications are hereby incorporated by reference: [1] Teodorescu DL, Miller SA, Jonnalagedda S. SurgiBox: An ultraportable system to improve surgical safety for patients and providers in austere settings. IEEE Xplore GHTC 2017 (accepted, pending publication); [2] Teodorescu DL, Nagle D, Hickman M, King DR. An ultraportable device platform for aseptic surgery in field settings. ASME J Medical Devices. J. Med. Devices 10(2), 020924 (May 12, 2016); [3] Published international PCT application number PCT/US17/42266 filed on Jul. 14, 2017 and titled “Ultraportable System For Intraoperative Isolation and Regulation of Surgical Site Environment”. [4] Published international PCT application number PCT/US2019/051502 filed on Sep. 17, 2019 and titled “Data analytics and interface platform for portable surgical enclosure”. [5] Published international PCT application number PCT/US2019/032148 filed on May 14, 2019 and titled “Sterile Sleeves for Portable Surgical Systems”.

What is claimed is:

1. A portable surgical system comprising:
(a). a flexible enclosure separating a surgical environment inside the enclosure from a user environment outside the enclosure, the enclosure comprising one or more areas of high optical clarity for viewing the inside of the enclosure;
wherein, while the surgical system is deployed in use, only the surgical site is included within the surgical enclosure, and the remainder of the patient body is essentially excluded from the surgical environment inside the enclosure;
(b). an environmental control system configured to supply air to the enclosure such as to create essentially sterile conditions inside the enclosure;
(c). one or more ports for accessing the surgical site; and
(d). an exhaust system configured to eliminate air from the enclosure,
wherein the exhaust system comprises one or more exhaust-channels disposed on the side and along the enclosure;
wherein the exhaust-channels comprise a plurality of holes disposed on one or more lines along the exhaust-channels, the holes being configured to allow air to flow from the enclosure into the exhaust-channels and further into the environment outside the enclosure.

2. The portable surgical system of claim 1, wherein the enclosure further comprises an air-supply-tube receiving air from the environmental control system and configured to create essentially uniform airflow over the surgical site.

3. The portable surgical system of claim 2, wherein the air-supply-tube is a flexible tube disposed above the surgical site and wherein the air-supply-tube comprises in whole or in part a plurality of perforations disposed along the air-supply-tube.

4. The portable surgical system of claim 3, wherein the distribution of the perforations along the air-supply-tube essentially follow an inverse of an elliptically shaped function or the positions of the perforations along the air-supply-tube (x₁, x₂, x₃, x₄, . . . xₖ) follow the specific set of mathematical expressions: x₁=Φ₁(V, d, D, ρ, k, L); x₂=Φ₂(V, d, D, ρ, k, L); x₃=Φ₃(V, d, D, ρ, k, L); . . . xₖ=Φₖ(V, d, D, ρ, k, L) described in the description.

5. The portable surgical system of claim 1, wherein the plurality of holes disposed along the exhaust-channels follow an inverse of an elliptically shaped function, or wherein the position of the holes along one or more of the exhaust-channels mirror the positions of the perforations along the air-supply-tube, or wherein the spacings between holes along one or more of the lines of the exhaust channels follows the same proportions as the spacings between perforations along the air-supply-tube.

6. The portable surgical system of claim 1, wherein the exhaust-channels comprise one or more exhaust-valves configured to adjust the airflow magnitude into the exhaust-channels and wherein the environmental control system is configured to adjust the airflow magnitude into the air-supply-tube via a supply-valve.

7. The portable surgical system of claim 1, wherein the exhaust-channels comprise one or more filters for filtering unwanted substances and materials from the air coming out of the exhaust-channels.

8. The portable surgical system of claim 6, comprising a plurality of sensors further comprising one or more of the following: pressure sensors disposed in the enclosure; pressure sensors disposed in the channels or the air-supply-tube; airflow sensors configured to measure airflow at various points inside the enclosure, in the channel or in the air-supply-tube.

9. The portable surgical system of claim 8, further comprising a control system configured to:
(a). receive from the sensors information about the enclosure environment;
(b). generate a sequence of action-steps leading to a desired enclosure environment, the actions steps comprising the controlling of airflow supply into the air-supply-tube and the controlling of exhaust airflow into exhaust-channels;
(c). control the airflow supply into the air-supply-tube and the exhaust airflow into exhaust-channels such as to attain the desired enclosure environment.

10. The portable surgical system of claim 3, wherein the air-supply-tube further comprises one or more flow-guides and wherein each of the flow-guides is disposed on top of a corresponding perforation such as to guide the flow of air from the air-supply-tube into the enclosure.

11. The portable surgical system of claim 10, wherein each of the flow-guides comprises a mini-valve configured to control the magnitude of the airflow through the corresponding perforation on which the flow-guides is disposed on.

12. The portable surgical system of claim 11, wherein the environmental control system is configured to control the mini-valves via electrically controlled actuators.

13. The portable surgical system of claim 12, further comprising a control system configured to:
(a). receive from the sensors information about the enclosure environment;
(b). generate a sequence of action-steps leading to a desired enclosure environment, the actions steps comprising the controlling of airflow supply into the air-supply-tube by controlling the mini-valves;
(c). control the airflow supply into the air-supply-tube, the exhaust valves and the mini-valves such as to attain the desired enclosure environment.

14. The portable surgical system of claim 12, wherein the exhaust-channels comprise one or more exhaust-valves configured to adjust the airflow magnitude into the exhaust-channels and wherein the environmental control system is configured to adjust the airflow magnitude into the air-supply-tube via a supply-valve.

15. The portable surgical system of claim 15, further comprising a control system configured to:
(a). receive from the sensors information about the enclosure environment;
(b). generate a sequence of actions leading to a desired enclosure environment, the actions comprising:
the controlling of airflow supply into the air-supply-tube by at least one of the following: controlling the mini-valves, controlling a supply-valve disposed in the environmental control system or on the air-supply-tube; and
the controlling of the exhaust-valves;
(c). control the airflow supply into the air-supply-tube, the exhaust valves and the mini-valves such as to attain the desired enclosure environment.

16. The portable surgical system of claim 1, further comprising a fluids and debris elimination-channel which is communicating with the enclosure via a set of openings;
wherein the elimination-channel is configured to enable the elimination of blood and bodily fluids from the enclosure;
wherein the elimination channel is disposed in a lower part of the portable surgical system such as to collect the blood and bodily fluids accumulated into the enclosure as a result of the surgical procedure.

17. The portable surgical system of claim 16, wherein the elimination channel further comprises one or more of the following: a valve, a suction device, a reservoir for accumulating and storing fluids and debris.

18. A portable surgical system configured to be used either for performing surgical procedures on the torso of a patient or on a limb of a patient, the system comprising:
(a). a flexible enclosure separating a surgical environment inside the enclosure from a user environment outside the enclosure, the enclosure comprising one or more areas of high optical clarity for viewing the inside of the enclosure;
wherein, while the surgical system is deployed in use for surgical procedures on the torso of a patient, only the surgical site is included within the surgical enclosure, and the remainder of the patient body is essentially excluded from the surgical environment inside the enclosure;
wherein, while the surgical system is deployed in use for surgical procedures on a limb of a patient, only the limb is included within the surgical enclosure
(b). an environmental control system configured to supply air to the enclosure such as to create essentially sterile conditions inside the enclosure;
(c). an exhaust system configured to eliminate air from the enclosure.

19. The portable surgical system of claim 18, wherein the exhaust system comprises one or more exhaust-channels disposed on the side and along the enclosure and wherein the exhaust-channels comprise a plurality of holes disposed along the exhaust-channels configured to allow air to flow from the enclosure into the exhaust-channels and further into the environment outside the enclosure.

20. A method of operating the portable surgical system of claim 15, the method comprising:
(a). measuring via the sensors a set of environmental parameters corresponding to an environmental status of the enclosure environment, the exhaust channels and the air-supply-tube;
(a). receiving at the environmental control system information about the environmental parameters measured in the enclosure, the exhaust channels and the air-supply-tube;
(b). analyzing the information received from the sensors regarding the environmental parameters;
(c). generating a sequence of actions leading to a desired environmental status of the enclosure environment;
(d). controlling the mini-valves, the supply-valves and the exhaust-valves according to the sequence of actions.
